(19) United States
(12) Patent Application Publication
(10) Pub. No.: US 2013/0131690 A1
(43) Pub. Date: May 23, 2013

(54) MEDICAL DEVICE FOR REMOVING CONCRETIONS

(71) Applicants: Frank Nagl, Karlsruhe (DE); Werner Mailänder, Grunbach (DE); Giorgio Cattaneo, Karlsruhe (DE); Kirsi Schüssler, Pforzheim (DE)

(72) Inventors: Frank Nagl, Karlsruhe (DE); Werner Mailänder, Grunbach (DE); Giorgio Cattaneo, Karlsruhe (DE); Kirsi Schüssler, Pforzheim (DE)

(73) Assignee: ACANDIS GMBH & CO. KG, Pfinztal (DE)

(21) Appl. No.: 13/698,769

(22) PCT Filed: May 18, 2011
(86) PCT No.: PCT/EP11/02483

§ 371 Date: Feb. 5, 2013

(30) Foreign Application Priority Data
May 18, 2010 (DE) 10 2010 020 869.8
Sep. 14, 2010 (DE) 10 2010 045 367.6

(51) Int. Cl. A61B 17/3207

(52) U.S. Cl. CPC A61B 17/3207
USPC 606/127

(57) ABSTRACT

A medical device for removing concretions from hollow organs of the body, with a functional element, which has a rotationally symmetrical lattice structure, and a device for holding a concretion in the structure, and a catheter for introducing the element into the body and removing it. The element can be converted from a compressed state in the catheter to an expanded state outside the catheter, in the expanded state the element is arranged distally from the catheter. The structure has a cutting area with webs, adapted so they at least partially pass radially through the concretion when the element is converted from the compressed to the expanded state. A holding element is connected on one hand to the structure and on the other hand to a guide wire arranged in the catheter, so that the holding element is deflected radially inwards in the expanded state of the functional element.

The invention relates to a medical device for removing concretions from hollow organs of the body according to the preamble of patent claim 1. A device of this type is known for example from WO 2006/031410 A2.

In practice, thrombectomy systems are used to treat thromboses, in particular in interventional neuroradiology. In this case, the thrombus or the blood clot triggering the thrombosis is mechanically removed from the blood vessel. A system of this type is described in WO 2006/031410 A2, cited in the introduction.

The known system comprises a catheter, which is adapted to supply a basket-like catch element. The basket-like catch element comprises a lattice structure, which can be converted from a compressed state into an expanded state. In the expanded state, the catch element is arranged distally from the catheter and has a rotationally symmetrical structure.

With the known system, the catch element is guided through the catheter to the treatment site. There, the catch element is released and expanded so that the basket-like structure of the catch element is formed. The catheter is also connected to an aspiration unit, such that a negative pressure can be generated in the region of the basket-like catch element, said negative pressure drawing the thrombus into the catch element. In this case, the lattice structure of the catch element lies against the vessel wall of the blood vessel to be treated. The thrombus is encapsulated in the catch element and can be drawn from the blood vessel via the catch element, wherein the lattice structure of the catch element slides along the vessel wall of the blood vessel.

Due to the contact between the catch element and the vessel wall of the blood vessel to be treated, the blood vessel wall is mechanically stressed. The vessel wall may thus become damaged. The movement of the catch element along the vessel wall intensifies this effect. Known systems, in particular the system according to WO 2006/031410 A2, cited in the introduction, generally have the disadvantage of subjecting the vessel walls of the hollow organ of the body to be treated to increased stress.

The object of the invention is to disclose a medical device for removing concretions from hollow organs of the body, with which the risk of complications caused by damage of vessel walls is reduced. Furthermore, a medical device for removing concretions from hollow organs of the body will also be disclosed, which has improved handling and reliability during use.

In accordance with the invention, this object is achieved by the subject matter of patent claim 1, and alternatively by the subject matter of the additional independent claim, patent claim 15.

The invention has the advantage that the anchoring of the concretion at the functional element is improved by the combination of the cutting region with the holding element, even in difficult conditions, for example if the concretion is transported by the functional element through strongly curved or expanding vessel cross sections. In this case, the holding element follows the concretion, even if the cutting region becomes disengaged, at least temporarily and/or in part, from the concretion. The holding function is thus maintained and the concretion remains fixed to the functional element.

In a preferred exemplary embodiment, the holding element forms a tongue-shaped cell with a free tip, which is arranged in a diamond-shaped lattice cell and/or mesh of the lattice structure.

The exemplary embodiment is a cell-in-cell arrangement, in which the outer cell, which surrounds the holding element, is diamond-shaped. In this case, the holding element is connected to the outer cell. The diamond shape of the outer cell improves the flexibility of the structure. The functional element is therefore particularly effectively adapted to expand in the concretion and to hold it from the inside out. A tensile force acting in the proximal direction is exerted onto the expanded functional element to detach and remove the concretion. This leads to an elongation of the outer cell due to the diamond shape. Due to the elongation of the outer cell, the holding element or the tongue-shaped cell arranged in the outer cell is elongated and lengthened. The lengthening of the holding element is accompanied by an elongation or deflection of the holding element radially outwardly.

In addition, the holding element or the tongue-shaped cell arranged in the outer cell is reinforced upon compression. A further effect of the elongation of the holding element lies in the fact that the holding element is closed in a scissor-like manner or the webs of the tongue-shaped cell arranged in the outer cell are moved toward one another. The concretion is thus clasped.

On the whole, the anchoring at the functional element of the concretion held from the inside out is improved by the diamond shape of the outer cell together with the cell-in-cell arrangement.

All cells of the lattice structure, that is to say not only the outer cells, but also the cells of the cutting region, may be diamond-shaped. The above-described kinematics of the lattice structure as well as the crimping behavior are thus further improved. The lattice structure with the diamond-shaped cells or meshes also has an increased radial force and is therefore particularly well-suited to cut or press deeply into the concretion during the expansion of the functional element released inside the concretion.

The tongue-shaped cell with the tip is arranged in the cylindrical lateral surface of the lattice structure in the expanded state of the functional element and in the rest position. During use, the tongue-shaped cell with the tip can be deflected radially outwardly and/or radially inwardly from the cylindrical lateral surface, in such a way that the tongue-shaped cell with the tip protrudes beyond the lateral surface of the lattice structure to hold the concretion.

Since the tongue-shaped cell is arranged in the cylindrical lateral surface of the lattice structure in the rest state, the functional element can crimp effectively and can be introduced easily and reliably into the hollow vessel through the catheter. In the vessel, the tongue-shaped cell can be deflected radially outwardly by a longitudinal curvature of the lattice netting, such that the tip of the tongue-shaped cell and therefore also a large part of the tongue-shaped cell itself or the complete tongue-shaped cell protrudes beyond the lateral surface to hold the concretion. The deflection of the tongue-shaped cell is further improved in that the lattice cell or mesh, in which the tongue-shaped cell is arranged, is more flexible than the lattice cells and/or meshes of the surrounding cutting region. If the tongue-shaped cell is arranged over the outer radius of the functional element in the event of longitudinal curvature, the tongue-shaped cell remains engaged with the concretion when the functional element follows a curvature of the vessel. If a plurality of tongue-shaped cells are distributed over the circumference of the functional element, the arrangement over the outer radius in the event of a longitudinal curvature, that is to say in the event of a curvature about an axis running substantially transverse to the longitudinal axis of the functional element, is achieved independently of the direction of curvature.

Furthermore, the tongue-shaped cell or tongue may additionally or alternatively be deflectable radially inwardly, in particular by the concretion, which, due to the cutting region of the lattice structure, which surrounds the tongue-shaped cell at least in some regions, moves into the lumen of the functional element. In this case, the concretion entrains the tongue-shaped cell, such that this is moved radially inwardly. The inwardly deflected tongue-shaped cell forms an abutment for the concretion, as a result of which the tensile or compressive forces (depending on the orientation and direction of movement of the tongue-shaped cell) can be transferred. The anchoring of the concretion in the functional element is thus improved.

In accordance with a preferred embodiment, the lattice cell or mesh in which the holding element is arranged is more flexible, at least in some regions and in particular completely, than the lattice cells and/or meshes of the cutting region that surround the holding element.

Due to the different flexibility of the lattice cell or mesh, in which the tongue-shaped cell is arranged, and the surrounding lattice structure of the cutting region, the effect that the tongue-shaped cell is deflected and also that the cutting region can penetrate into the tissue of the concretion is improved, whereby the desired change in the relative position between the tongue-shaped cell and the cutting region is set.

The flexibility of the holding element relative to the surrounding cells or meshes can be influenced in a preferred embodiment in that the lattice cell or mesh, in which the holding element is arranged, is smaller than, the same size as, or larger than, the surrounding lattice cells or meshes of the cutting region.

In particular if the lattice cell or mesh with the holding element is larger than the surrounding lattice cells or meshes of the cutting region, the axial deformability of the larger cell is then increased. This leads to the fact that a cell series, which consists of larger cells, bends in an improved manner and therefore has increased flexibility. In combination with the holding element, this leads to the fact that the holding element is displaced outwardly when the bend, in particular the longitudinal bend of the functional element, is encountered. The size of the cell relates to the width or height thereof. Alternatively, the size may also relate to the cell area.

All holding elements can be oriented in the distal direction in such a way that the tips point in the distal direction.

The ratio of the web length of the lattice cell or mesh with the holding element to the web length of the surrounding lattice cells or meshes of the cutting region may be at least 110%, in particular at least 120%, at least 130%, at least 140%, at least 150%, at least 175%, or at least 200%. The holding elements may have webs of which the web width is at most 150%, in particular at most 120%, at most 110%, at most 90%, at most 80%, at most 70%, at most 60%, at most 50%, or at most 40%, of the web width of the surrounding lattice cells or meshes of the cutting region. In a further preferred exemplary embodiment, the ratio of the web width of the webs of a lattice cell or mesh with a holding element to the web width of the surrounding lattice cells or meshes of the cutting region is at most 200%, in particular at most 175%, at most 50%, at most 20%, at most 110%, at most 90%, at most 80%, at most 70%, at most 60%, or at most 50%, of the web width.

In a further preferred exemplary embodiment, at least one annular segment with one or with a plurality of holding elements and at least one annular segment without holding element are arranged in the longitudinal direction of the lattice structure. The ratio of the number of cells of an annular segment with holding element to the number cells of an annular element without holding element can be at most 3:4, in particular 2:3, at most 1:2, or at most 1:3. The ratio of the number of annular elements with at least one holding element to the number of annular elements without holding element may be at most 1:3, in particular at most 1:2, at most 2:3, at most 1:1, at most 3:2, at most 2:1, at most 3:1, or at most 4:1.

The annular segments with at least one holding element and the annular segments without holding element may alternate in the longitudinal direction of the lattice netting. A preferred pattern is the alternating arrangement of an annular segment with one or more holding elements and an annular segment without holding elements. Other alternating patterns are possible, such as:

one annular segment with at least one holding element/two annular segments without holding element, or vice versa,

one annular segment with at least one holding element/three annular segments without holding element, and vice versa, and

two annular segments each with at least one holding element/three annular segments each without holding element, and vice versa.

In terms of the ratio of the number of annular segments with at least one holding element and an annular segment without holding element, the following specific embodiments are conceivable. At least 1, in particular at least 2, at least 3, at least 4, at least 5, or at least 6, annular elements with at least one holding element or annular elements without holding element may be provided in the longitudinal direction of the device. At most 6, in particular at most 5, in particular at most 4, in particular at most 3, in particular at most 2, or in particular at most 1, annular element(s) with at least one holding element or annular element(s) without holding element may be provided in the longitudinal direction of the device. The aforementioned upper and lower limits may be combined with one another to form ranges.

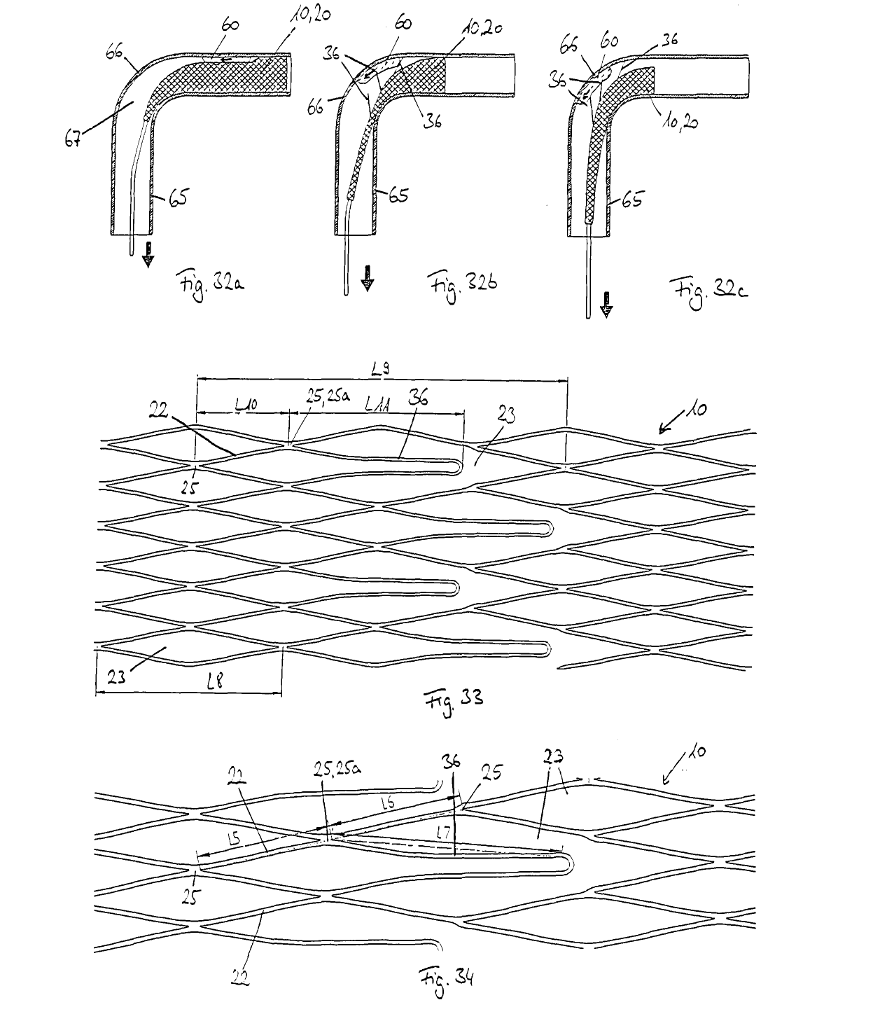

Lattice cells or meshes each with at least one holding element and lattice cells or meshes without holding element may be arranged within an annular segment offset from one another in the axial direction (for example FIGS. 33 to 35), in particular if the number of cells of the annular element with at least one holding element deviates from the number of cells of the annular element without holding element, for example due to the different size. The connection line of the centers of gravity or midpoints of the lattice cells defines a zigzag shape in the circumferential direction.

A further object of the invention is based on the concept of specifying a medical device for removing concretions from hollow organs of the body, said medical device having a functional element, which has a rotationally symmetrical lattice structure and a means for holding a concretion in the lattice structure, and having a catheter for introducing the functional element into the body and for removing it therefrom, wherein the functional element can be converted from a compressed state in the catheter into an expanded state outside the catheter, in which the functional element is arranged distally from the catheter. The lattice structure has a cutting region with webs, which, during use, at least partially radially penetrate the concretion when the functional element is converted from the compressed state into the expanded state. The holding means comprises at least one holding element, which is connected to the lattice structure and also to a guide wire arranged in the catheter, in such a way that the holding element is deflected radially inwardly in the expanded state of the functional element.

The invention is based on a concept that is fundamentally different from the known thrombectomy system, in which the catcher element lies against the vessel wall and the thrombus is sucked into the proximally arranged catcher element by aspiration. Rather, due to the cutting region of the lattice structure, it is possible with the device according to the invention to expand the lattice structure, or generally the functional element, within the concretion. The webs of the cutting region penetrate the concretion in the radial direction during this process. The webs of the cutting region can anchor themselves in the concretion, so that contact with the vessel wall of the hollow organ of the body is avoided. The stress to which the vessel wall is subjected during use of the device is thus reduced. Furthermore, the device according to the invention can be handled more easily and demonstrates improved reliability during use. In particular, the reliability during use is increased by the holding elements connected to the guide wire. Whilst the webs of the cutting region advance radially into the concretion, the holding elements are deflected radially inwardly and thus form a connection between the guide wire and the plane of the wall of the lattice structure. Due to the connection to the guide wire, the holding elements are forcibly driven during expansion. The radially inwardly deflected holding elements ensure an anchoring of the concretion in the functional element, even if the webs of the cutting region fully penetrate the concretion. The concretion is thus fixed securely within the functional element and can be removed easily from the hollow organ of the body. The effective cross section of the functional element is increased by the holding elements.

The invention functions in principle with a single holding element. The efficacy of the invention is increased by the arrangement of a plurality of holding elements. The holding element can be connected to the guide wire fixedly or so as to be axially longitudinally displaceable. In the case of a fixed connection between the holding element and the guide wire, it is advantageous if the holding element has increased flexibility or resilience so as to compensate for the change in length and diameter of the functional element during expansion. The advantage of a fixed connection between the holding element and the guide wire lies in the fact that the degree of expansion of the functional element can be set in some regions, in particular over points at the connection point between the holding element and the lattice structure. Alternatively, the change in length and diameter of the functional element during expansion can be achieved by an axially displaceable mounting of the holding element on the guide wire. Due to the axially displaceable mounting, the functional element can also be adapted individually to the structure of the concretion.

In a preferred embodiment of the medical device according to the invention, the holding element has a tip, which is connected to the guide wire. Specifically, the holding element may extend from the circumferential plane of the lattice structure to the guide wire in the expanded state of the functional element.

The tip may have an eyelet or a loop or a sliding sleeve, through which the guide wire is guided. A construction of the tip of this type can be produced in a particularly simple manner. A simple and reliable axially displaceable connection between the holding element and the guide wire is enabled as a result of the eyelet, the loop or the sliding sleeve.

The tip may further have an enlarged contact area, in particular formed in a spoon-like manner. The contact area improves the anchoring of the holding element within a concretion. Due to the deflection of the holding element in the expanded state of the functional element, a substantially radial orientation of the contact area of the tip of the holding element is advantageously produced. The contact area thus cooperates with the concretion during movement of the concretion and thus increases the fixing of the concretion within the functional element.

The following embodiments are also disclosed in conjunction with the device according to claim 1, in which the connection between the holding element or the holding elements and the guide wire is not compulsory.

The holding element is preferably substantially V-shaped. The holding element comprises two spokes, which are each connected to at least one web of the cutting region. A V-shaped structure of this type with spokes is easily possible within the scope of production of medical lattice structures. The holding element can thus be produced or integrated into the medical lattice structure in a simple manner.

The spokes of the holding element may have a lower mechanical rigidity, in particular a smaller width and/or thickness, than the webs of the cutting region. Generally, the holding element has increased flexibility or reduced mechanical rigidity compared to the webs of the cutting region. For example, the increased flexibility can be set by the width or thickness of the spokes. Alternatively or additionally, the increased flexibility of the holding element or of the spokes can be influenced by material properties. Further factors that may influence the flexibility of the holding element or of the spokes of the holding element compared to the webs of the cutting region include, for example, the pivot angle between the spokes of the holding element and the webs of the cutting region and/or the length of the spokes compared to the webs of the cutting region. In all possible configurations, the increased flexibility or reduced mechanical rigidity results in an improved deflection behavior of the holding element. Specifically, the holding element is adapted in such a way that, when the concretion is penetrated by the functional element, the holding element is deflected radially inwardly. The holding element can thus be deflected not only by the connection to the guide wire, but also by the pressure of the concretion material. By contrast, the webs of the cutting region are stiff, such that the webs penetrate the material of the concretion.

In accordance with a preferred embodiment, the lattice structure comprises at least one catcher element, which protrudes radially outwardly beyond the lattice structure in the expanded state. The catcher element has the advantage that the parts of the concretion located between the lattice structure and the vessel wall are picked up by the catcher element when the functional element is withdrawn into the catheter.

The holding element may extend in the longitudinal axial direction of the functional element in the compressed state of the functional element. The holding element is thus effectively integrated into the lattice structure, such that a relatively small, compressed cross-sectional diameter of the functional element can be set. However, it is not ruled out that the holding element extends in the circumferential direction in the compressed state of the functional element.

A plurality of holding elements are advantageously provided. The anchoring function of the functional element with respect to the concretion to be removed is thus further improved. The plurality of holding elements may have different lengths. Holding elements that are connected to the webs of the cutting region substantially in the same axial portion of the rotationally symmetrical lattice structure can thus be connected at different points to the guide wire, whereby the ease of handling and the reliability during use of the medical device are increased. Furthermore, it is possible to ensure as a result of the different lengths of the plurality of holding elements that the guide wire runs in a centered manner within the functional element in the expanded state of the functional element.

A plurality of holding elements may also be provided, which are arranged adjacently in the circumferential direction at an axial end of the functional element. The plurality of holding elements may be connected to the guide wire so as to form a net-like closure of the functional element. Specifically, the plurality of holding elements may be arranged in the same axial end portion of the lattice structure or of the functional element. The tips of the holding elements may be connected to the guide wire, such that the axial end of the lattice structure remains closed during the expansion of the functional element. A completely closed lattice structure can thus be formed during the expansion of the functional element, thus improving the encapsulation of a concretion.

In a further preferred embodiment of the medical device, the guide wire comprises at least one delimitation element. In the expanded state of the functional element, the delimitation element may delimit an axial movement of the holding element, in particular of the tip. Due to the delimitation of the axial movement of the tip of the holding element, the possible expansion of the functional element is also delimited. The maximum possible expansion of the functional element can thus be set by a corresponding arrangement of the delimitation element. For example, the maximum possible expansion of the functional element may be set in such a way that the functional element does not contact the vessel wall of the hollow organ of the body to be treated. Damage to the vessel wall can thus be effectively avoided.

The delimitation element may comprise a groove, which is formed in the guide wire, or a sleeve, which is fixedly connected to the guide wire. A design of this type of the delimitation element can be produced in a particularly simple manner.

The guide wire may comprise a flexible element. The flexible element may be adapted in such a way that the guide wire can be longitudinally axially lengthened. The flexible element can compensate for the change in length, in particular the shortening of the functional element or the lattice structure during the expansion. A change in length or shortening of the lattice structure of this type is generally referred to as “foreshortening”. The flexible element may also have a bias, such that the expansion when the functional element is released from the catheter is assisted by the bias of the flexible element of the guide wire. The guide wire is preferably fixedly connected to the functional element. In particular, the guide wire can be fixedly connected to a distal end and/or a proximal end of the functional element.

In accordance with a further preferred embodiment of the medical device according to the invention, the functional element has at least one displacement element, which is arranged flush in the lattice structure with a straight-lined arrangement of the functional element. With a longitudinally curved arrangement of the functional element, the displacement element is deflected radially outwardly. Specifically, the displacement element is arranged flush in the plane of the wall of the lattice structure, irrespective of whether the lattice structure adopts the compressed state or the expanded state. The flush arrangement of the displacement element is generally implemented in the straight-lined arrangement of the functional element. This means that a longitudinal axis of the functional element or of the rotationally symmetrical lattice structure extends in a straight line. The displacement element is thus arranged flush in the lattice structure if the functional element or the lattice structure is substantially cylindrical. By contrast, with a curvature of the functional element in the longitudinal direction, the displacement element is displaced in a direction pointing radially outwardly. In the case of the longitudinal curvature, the functional element or the lattice structure leaves the cylinder shape and converts into a curved shape, similar to a pipe curvature.

The displacement element may be arranged on the side of the lattice structure that has the greater radius of curvature. The displacement element is preferably adapted in such a way that a deflection then only occurs if the displacement element is arranged on the side of the lattice structure that has the greater radius of curvature based on the longitudinal axis of the lattice structure.

Due to the curvature of the functional element or the lattice structure, the displacement element is deflected radially outwardly. With the arrangement of the functional element in vessel curvatures, there is a risk that a clearance is formed between a vessel wall and the lattice structure such that concretion particles may detach from the functional element. This is reduced by the displacement element, which is deflected radially outwardly in a vessel curvature and thus projects into the clearance formed. The reliable removal of a concretion from a hollow organ of the body is thus improved.

The invention will be explained in greater detail hereinafter on the basis of exemplary embodiments, with reference to the accompanying, schematic drawings, in which:

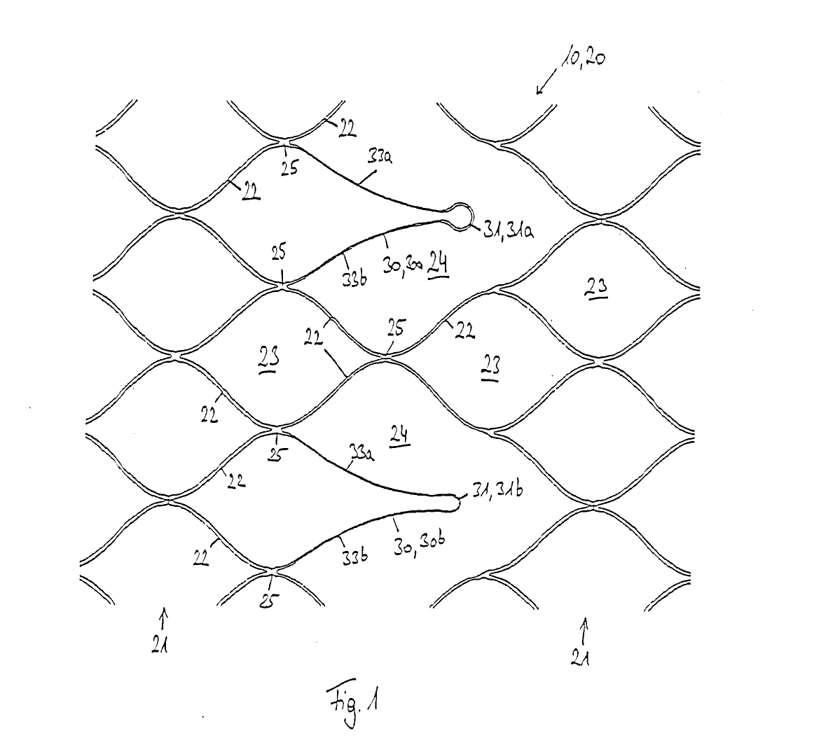

FIG. 1 shows a plan view of a portion of the lattice structure of a functional element of the medical device according to the invention in accordance with a preferred exemplary embodiment;

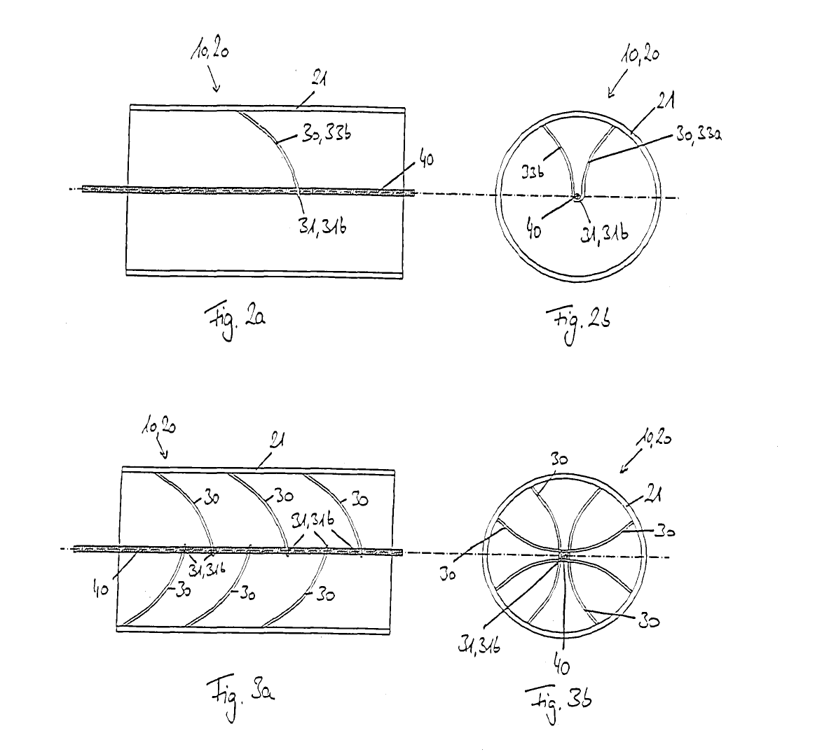

FIG. 2a shows a side view of a functional element of the medical device according to the invention in accordance with a preferred embodiment;

FIG. 2b shows a cross section of the functional element according to FIG. 2a;

FIG. 3a shows a side view of a functional element of the medical device according to the invention in accordance with a further preferred exemplary embodiment;

FIG. 3b shows a cross section of the functional element according to FIG. 3a;

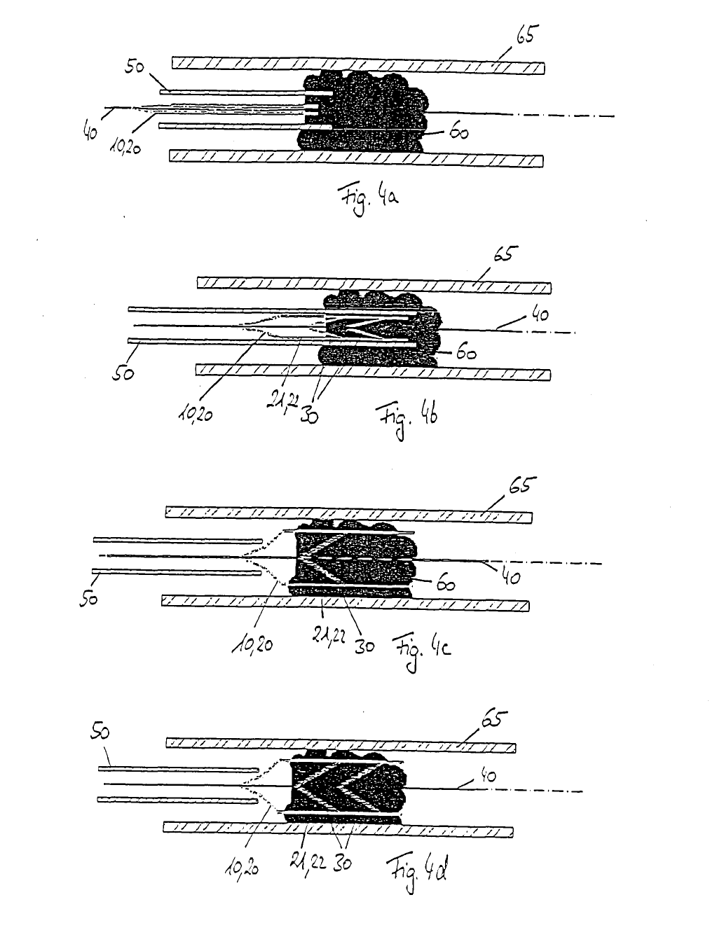

FIGS. 4a-4b each show a side view of the medical device according to the invention in accordance with a preferred exemplary embodiment in various phases of the release of the functional element in a blood vessel;

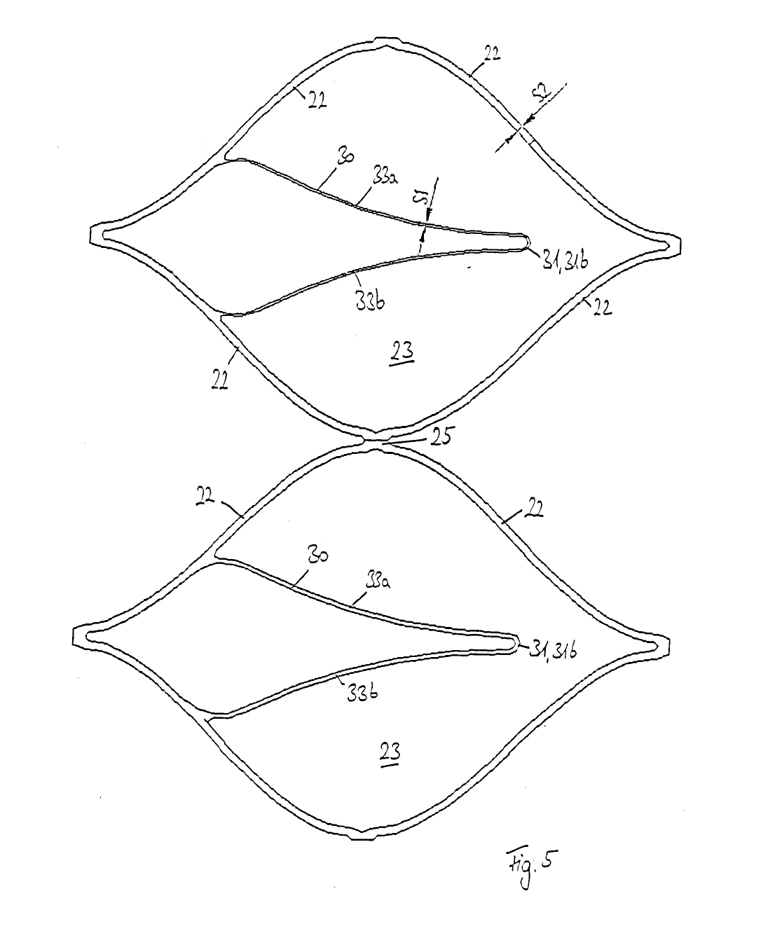

FIG. 5 shows a plan view of a detail of a lattice structure of the functional element of the medical device according to the invention in accordance with a preferred exemplary embodiment;

FIGS. 6-9 each show a detailed view of a cell area of the structure of the functional element of the medical device according to the invention in accordance with a preferred exemplary embodiment;

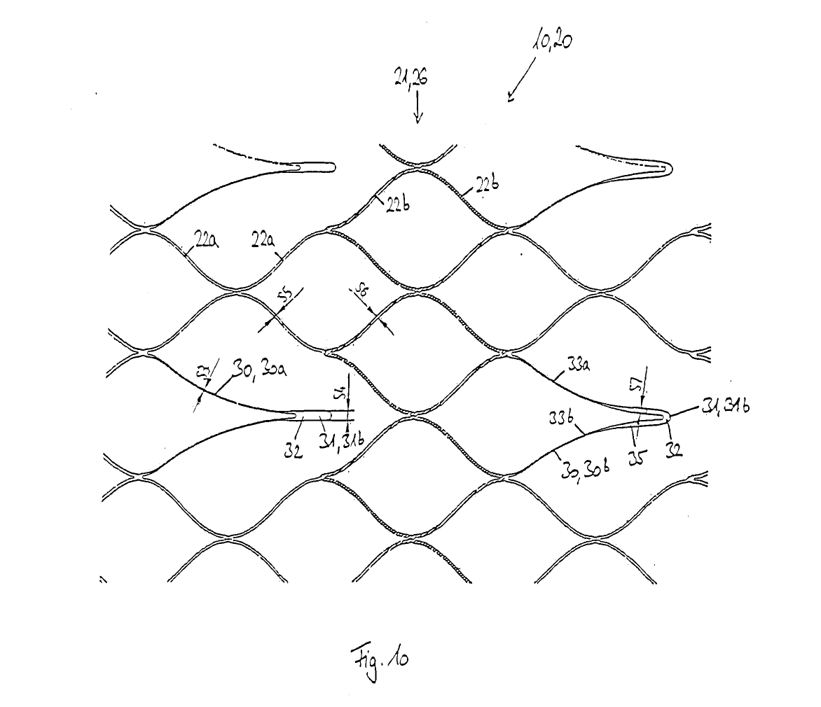

FIG. 10 shows a plan view of a lattice structure of the functional element of the medical device according to the invention in accordance with a further preferred exemplary embodiment;

FIGS. 11a-11d each show a development of a functional element of the medical device according to the invention in accordance with one of the preferred exemplary embodiments;

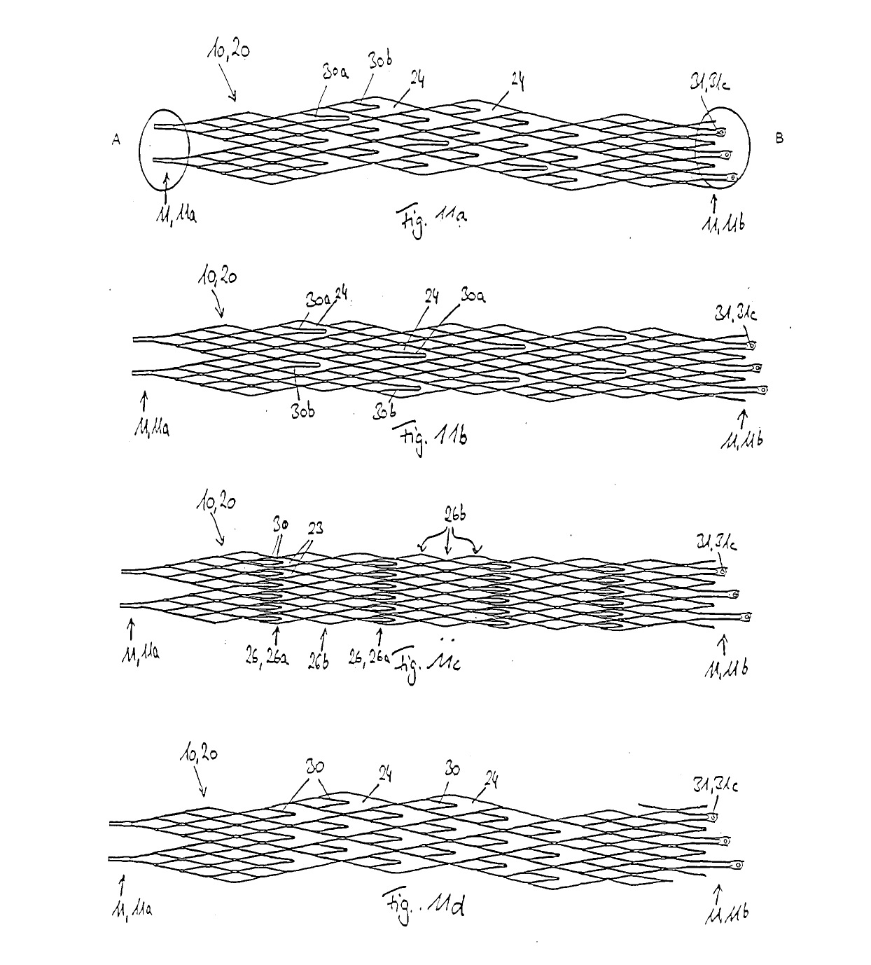

FIG. 12a shows a side view of a functional element of the medical device according to the invention in accordance with a further preferred exemplary embodiment;

FIG. 12b shows a cross section of the functional element according to FIG. 12a;

FIG. 13 shows a side view of a functional element of the medical device according to the invention in accordance with a further preferred exemplary embodiment;

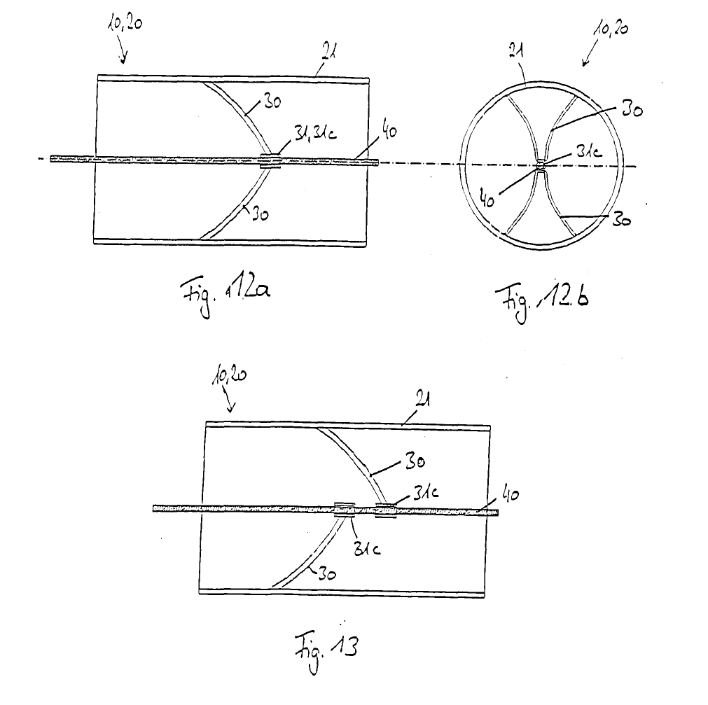

FIG. 14 shows a plan view of a lattice structure of the functional element of the medical device according to the invention in accordance with a further preferred exemplary embodiment;

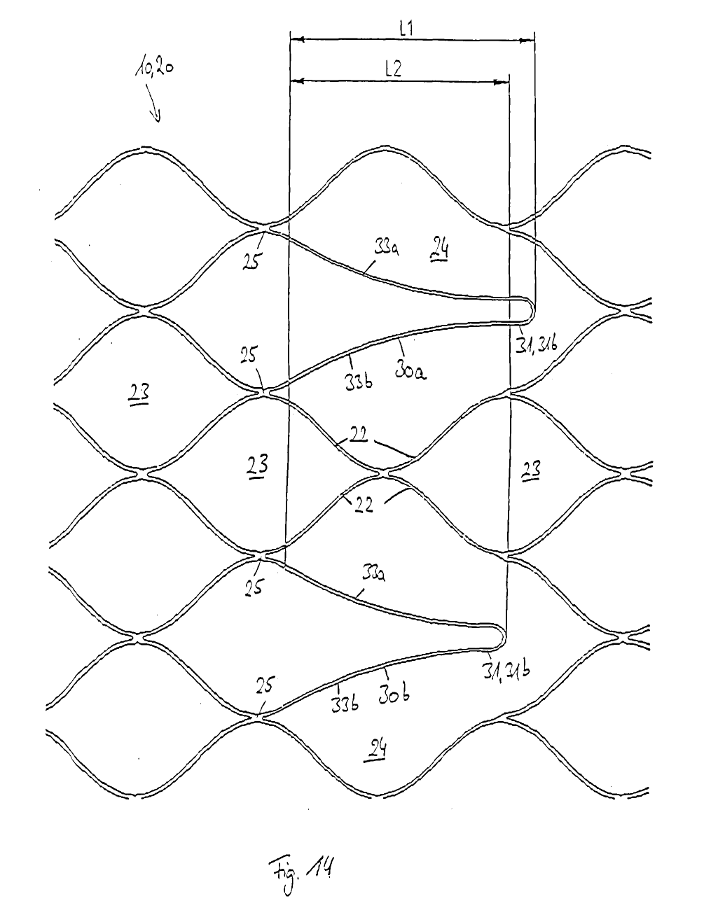

FIG. 15a shows a side view of a functional element of the medical device according to the invention in accordance with a further preferred exemplary embodiment;

FIG. 15b shows a cross section of the functional element according to FIG. 15a;

FIGS. 16a-16c each show a development of a functional element of the medical device according to the invention in accordance with a preferred exemplary embodiment;

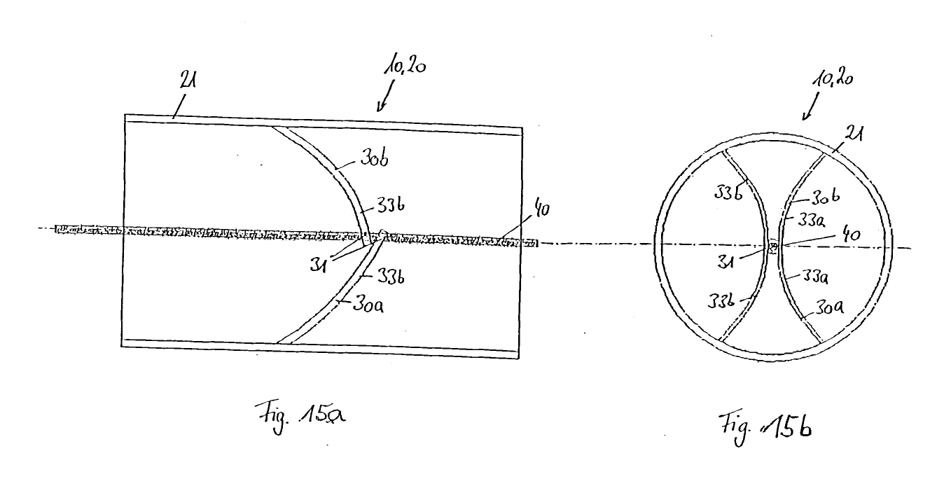

FIG. 17a shows a development of a lattice structure of the functional element of the medical device according to the invention in accordance with a preferred exemplary embodiment;

FIG. 17b shows a cross section of the functional element according to FIG. 17a;

FIG. 18a shows a development of a lattice structure of the functional element of a medical device according to the invention in accordance with a further preferred exemplary embodiment;

FIG. 18b shows a cross section of the functional element according to FIG. 18a;

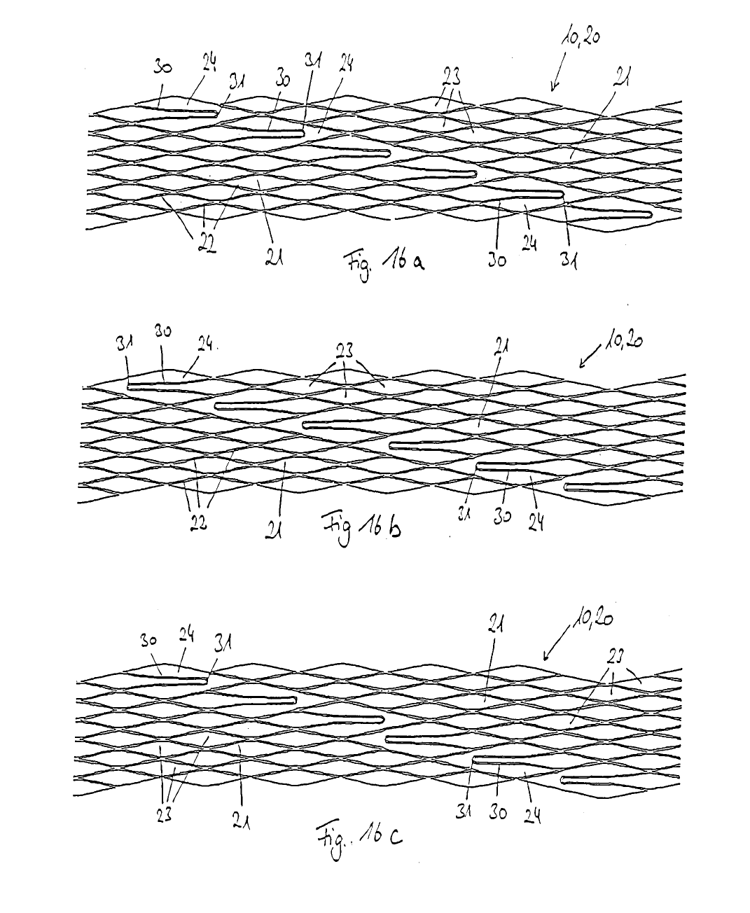

FIG. 19a shows a development of a lattice structure of the functional element of the medical device according to the invention in accordance with a further preferred exemplary embodiment;

FIG. 19b shows a cross-sectional view of the functional element according to FIG. 19a;

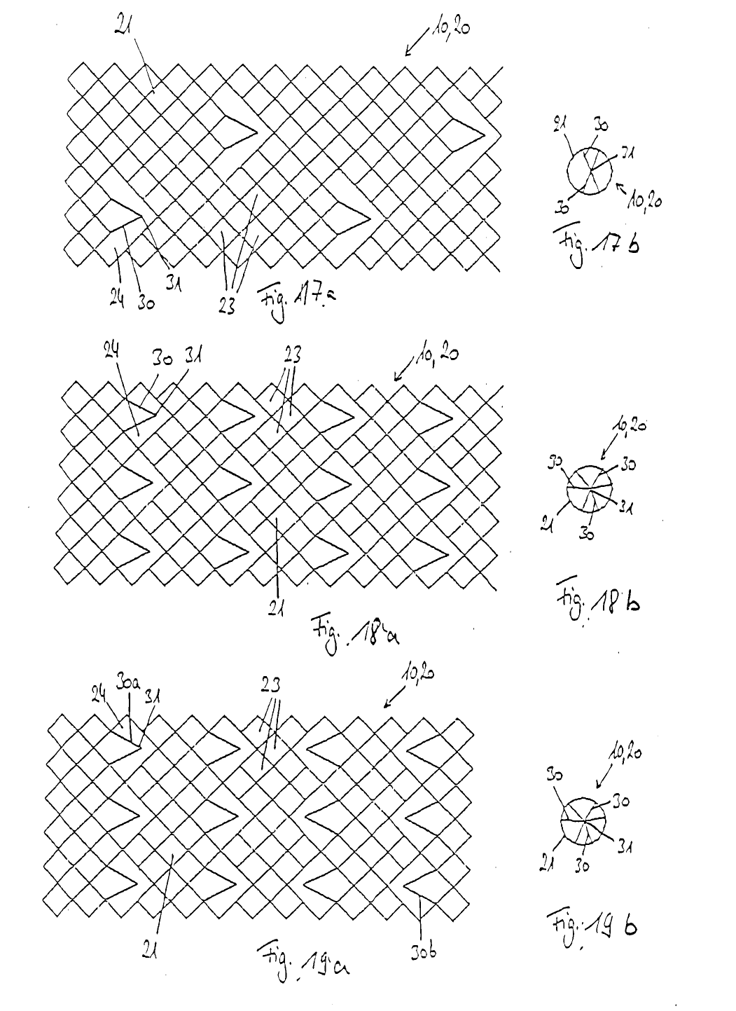

FIGS. 20a-20d each show a side view of a functional element of the medical device according to the invention in accordance with a preferred exemplary embodiment during use;

FIG. 21a shows a side view of a functional element of the medical device according to the invention in accordance with a preferred exemplary embodiment in a partly expanded state;

FIG. 21b shows a side view of the functional element of the medical device according to the invention according to FIG. 21a in a fully expanded state;

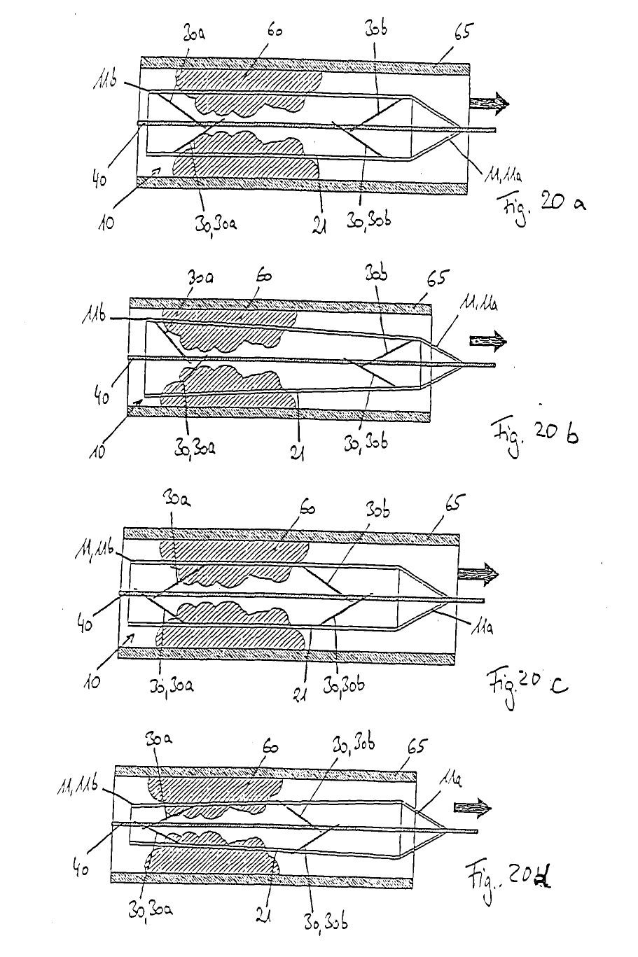

FIG. 22a shows a side view of a functional element of the medical device according to the invention in accordance with a further preferred exemplary embodiment in the partly expanded state;

FIG. 22b shows a side view of the functional element of the medical device according to the invention according to FIG. 22a in a fully expanded state;

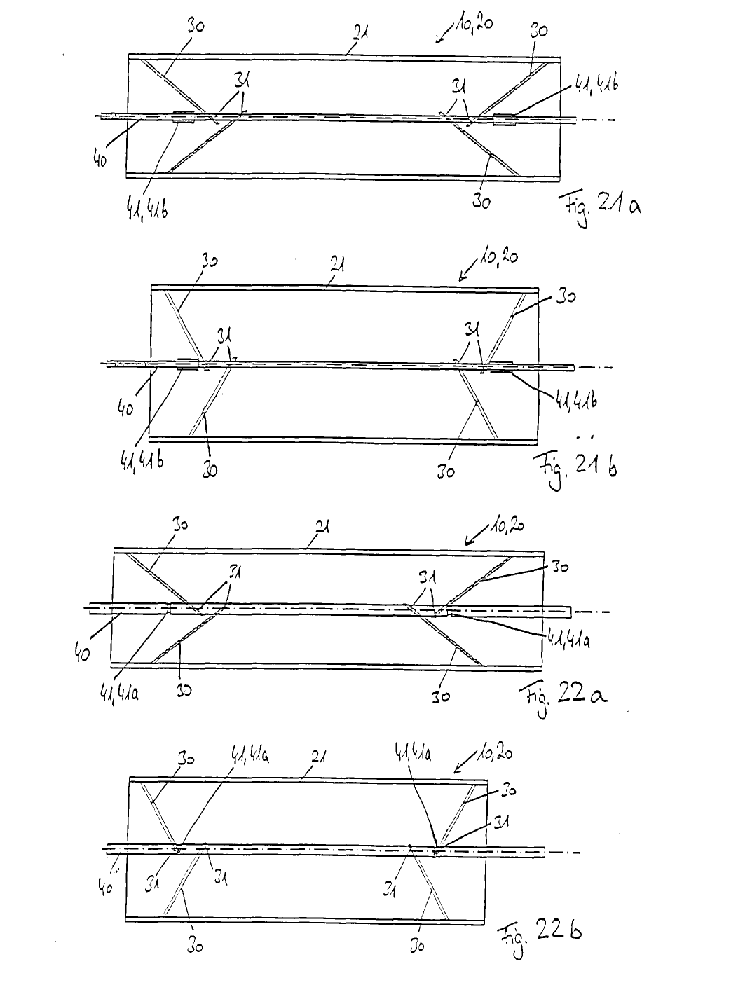

FIG. 23a shows a side view of a functional element of the medical device according to the invention in accordance with a further preferred exemplary embodiment in a partly expanded state;

FIG. 23b shows a side view of the functional element of the medical device according to the invention according to FIG. 23a in a fully expanded state;

FIG. 24a shows a side view of the medical device according to the invention in accordance with a further preferred exemplary embodiment in a partly expanded state of the functional element; and

FIG. 24b shows a side view of the device according to FIG. 29a in a fully expanded state of the functional element.

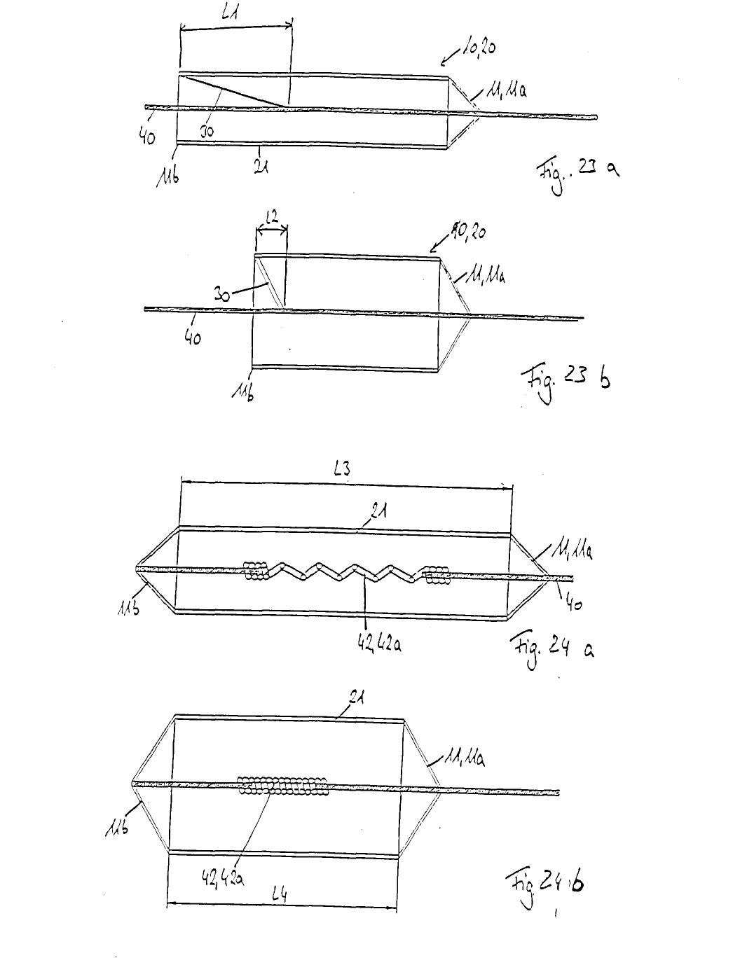

FIG. 25a shows a schematic side view of the device in accordance with an exemplary embodiment according to the invention with radially outwardly deflected catcher elements;

FIG. 25b shows a cross section through the device according to FIG. 25a;

FIG. 25c shows a development of the lattice structure of the device according to FIG. 25a;

FIGS. 26a-26c show a device in accordance with a further exemplary embodiment according to the invention with catcher elements during use in various stages of the removal of a concretion from a vessel;

FIG. 26d shows a cross section through the device according to FIG. 26c and of the vessel in which the device is arranged;

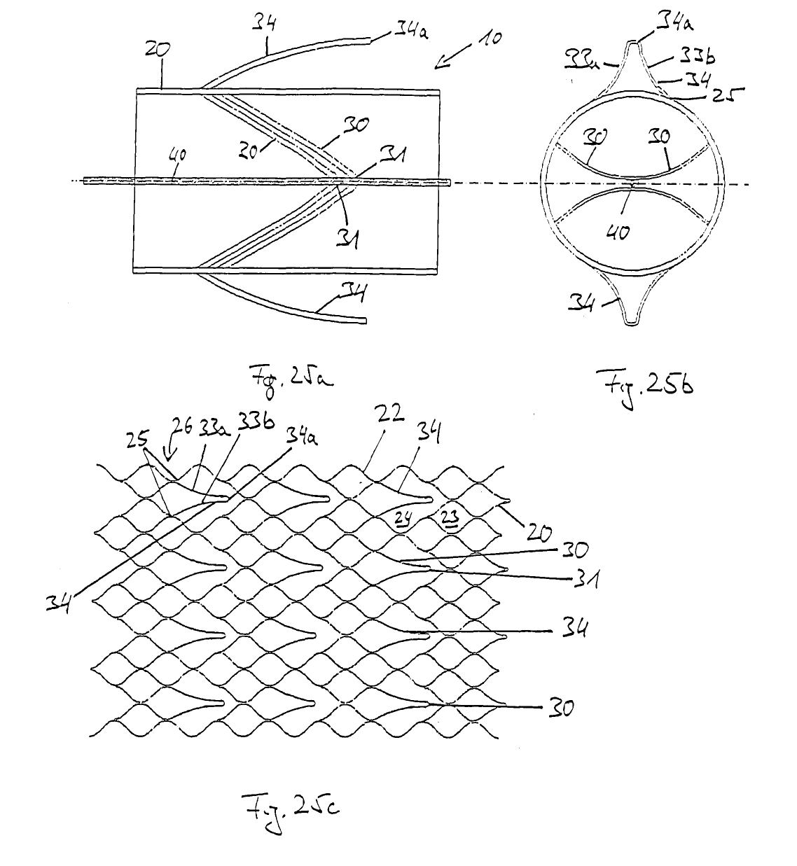

FIGS. 27a, 27b show a plan view of various variants of the catcher element;

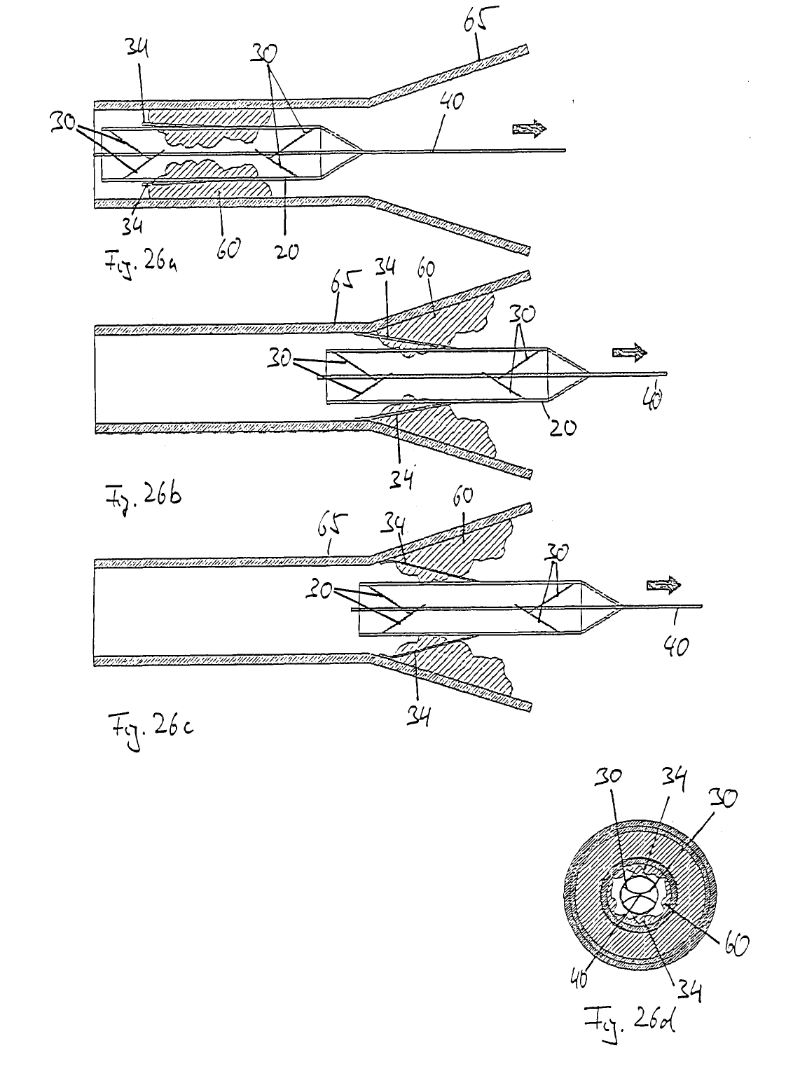

FIGS. 28a, 28b show side views of various variants of the catcher element with a curved free end;

FIG. 29 shows a plan view of a catcher element in accordance with a further exemplary embodiment with various functional portions;

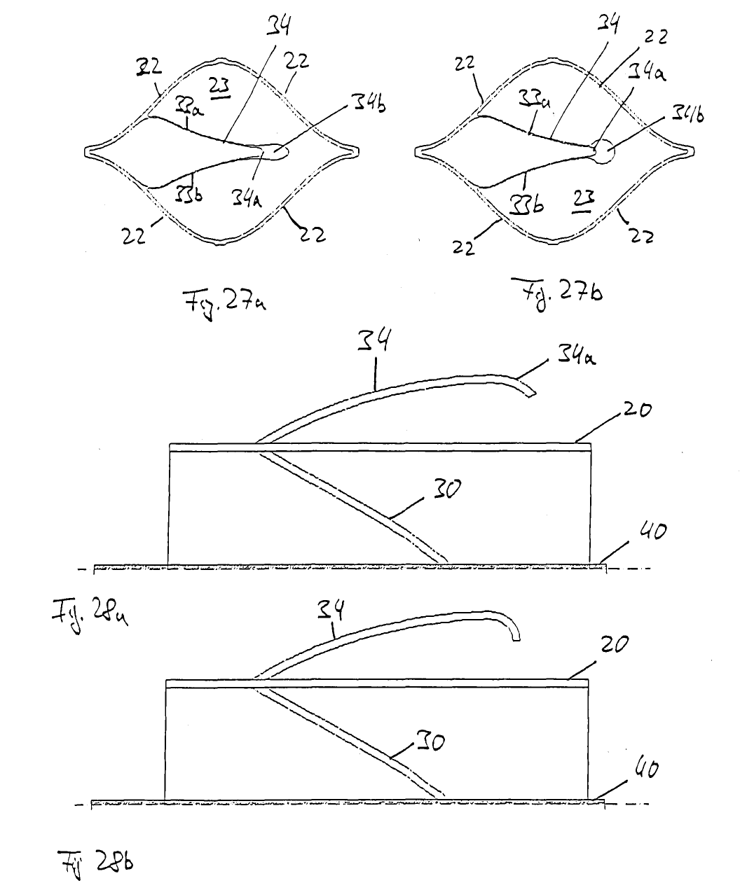

FIGS. 30a-30d show side views of a device in accordance with an exemplary embodiment according to the invention with a catcher element in various production stages;

FIG. 31 shows a plan view and side view of a mandrel for shaping the catcher element;

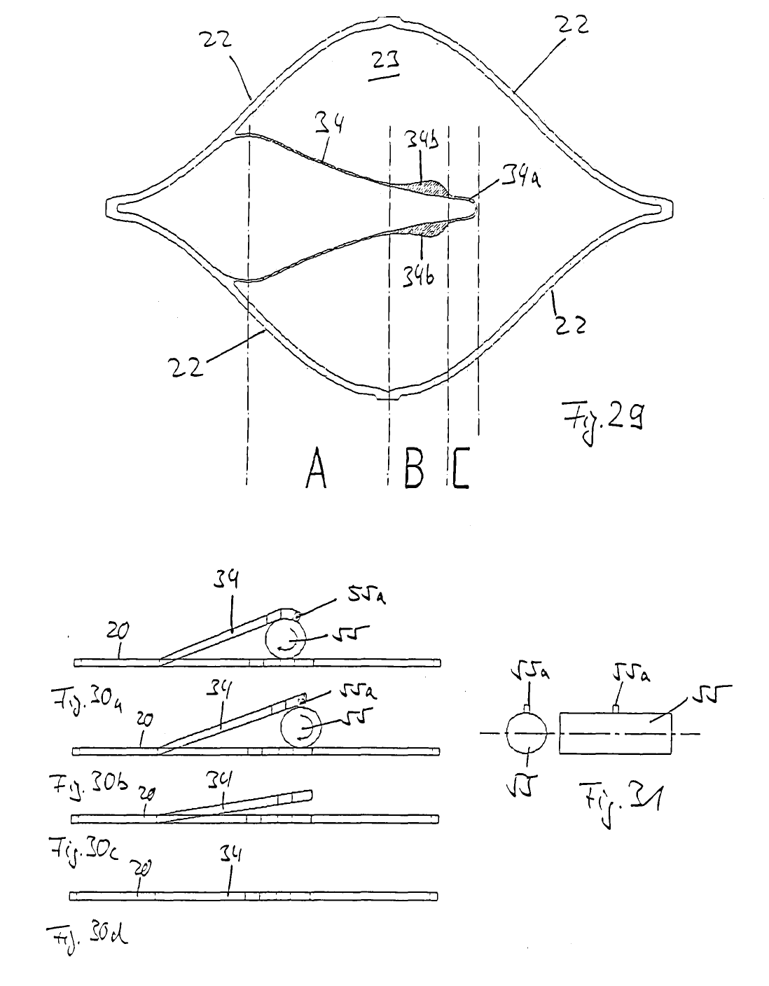

FIGS. 32a-32c show side views of a device in accordance with an exemplary embodiment according to the invention with displacement elements in different stages during the removal of the concretion;

FIG. 33 shows a development of the lattice structure of the functional element according to FIGS. 32a-32c;

FIG. 34 shows a detailed view of the development according to FIG. 33; and

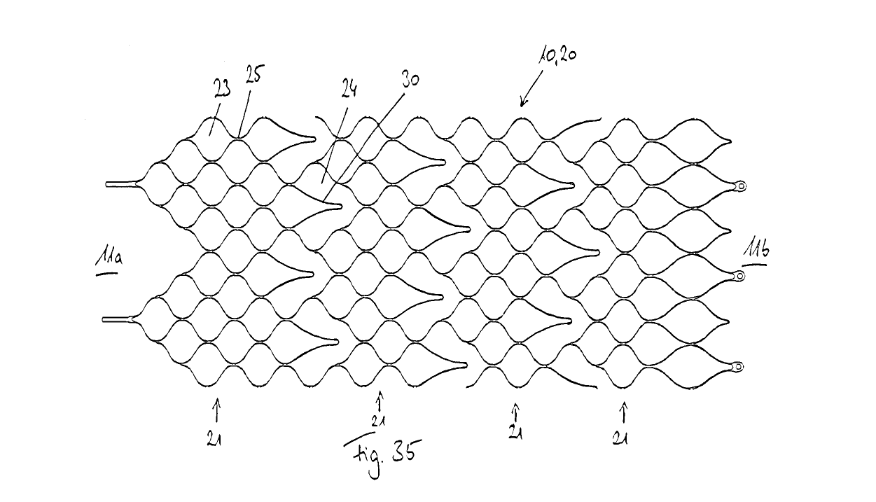

FIG. 35 shows a development of a functional element of the device according to the invention in accordance with a preferred exemplary embodiment.

The following embodiments relate both to the device in which the holding element 30 is connected to the guide wire, and to the device in which the holding element 30 is not connected directly to the guide wire, but merely to the lattice netting. In particular, the design of the lattice netting as well as the shape and operating principle of the holding element, as described hereinafter, also relate to the device according to claim 1.

The device described in the following exemplary embodiments generally has a functional element 10, which can be introduced into a hollow organ of the body, in particular a blood vessel 65, and can be expanded in the hollow organ of the body or blood vessel 65. To this end, the medical device has a catheter 50, in which the functional element 10 can be arranged in the compressed state so as to be longitudinally displaceable. In the expanded state, that is to say during the treatment of a hollow organ of the body or blood vessel 65, the functional element is arranged outside the catheter 50, in particular distally outside the catheter 50.

In this context, it is noted that the medical directional indications “distal” and “proximal” are based on a reference point in the form of the user of the medical device. Distally arranged elements or regions are therefore distanced further from the user of the medical device than proximally arranged elements or regions.

So as to enable a radial cross-sectional change when converting from the compressed state into the expanded state, the functional element 10 has a lattice structure 20. A portion of the lattice structure 20 is illustrated by way of example in FIG. 1. The lattice structure 20 comprises webs 22, which form a cutting region 21. The webs 22 are interconnected here and there. Node points 25 are formed at the connection points of the webs 22.

The webs 22 define cells 23 and also meshes 24. Within the scope of the application, lattice openings that are defined on all sides by a respective web 22, are referred to as cells. In other words, a cell 23 has four sides, which are each formed by a single web 22. By contrast, lattice openings comprising at least one defining side, which has at least two webs 22, are denoted as meshes 24. In this case, the at least two webs 22, which form one side of the mesh 24, run substantially in the same direction.

As a result, the lattice openings formed by meshes 24 are larger than the lattice openings formed by cells 23. Both the lattice openings formed by meshes 24 and those formed by cells 23 have a generally diamond-shaped contour. The term “diamond-shaped” is not to be understood strictly geometrically, but also includes the geometries illustrated for example in FIG. 1, in which the sides of the lattice openings are formed by webs curved in an S-shaped manner. Other shapes of the lattice openings are possible. Generally, the webs 22 of the cells 23 or of the meshes 24 are arranged at an incline with respect to the longitudinal direction of the functional element 10. In conjunction with the cutting function of the webs, the inclined arrangement has the advantage that the webs penetrated into the concretion during use provide an improved resistance when the concretion is removed from the vessel together with the functional element 10 compared to a situation in which the webs 22 are aligned parallel for example to the longitudinal direction of the functional element.

The cell angle is large so that the structure is flexible and also so that the cell deforms to a greater extent during conversion from the compressed state into the expanded state. Due to the deformation, the concretion can be better captured by the cells. The concretion is tucked in so to speak by the cells. The tip angle in the fully expanded state is greater than 70°, in particular greater than 80°, in particular greater than 90°, in particular greater than 110°, in particular greater than 120°, and in particular greater than 130°. So as to increase the resistance of the lattice structure 20 in the concretion, it is further advantageous to provide relatively many cells 23 or meshes 24, which cut into the concretion. For example, more than 4, in particular more than 6, more than 8, or more than 12, cells may be provided per annular segment of the functional element 10 or of the lattice structure 20. The lattice structure 20 comprises more than 2 annular segments, in particular more than 3 annular segments, in particular more than 4 annular segments, and in particular more than 5 annular segments, in the longitudinal direction of the functional element 10. The annular segments, which form the cutting region, are located in the same cylindrical lateral surface of the lattice structure 20.

The webs 22 or the cells 23 of the lattice structure 20 form the cutting region 21 of the lattice structure 20. The web width in the region of the cutting elements or of the webs 22 and of the cells 23 and/or the web width of the holding elements is small. The cutting function of the cutting elements is thus promoted. The web width of the cutting elements and/or of the holding elements may be less than 50 μm, in particular less than 40 μm, in particular less than 35 μm, in particular less than 30 μm, in particular less than 25 μm, in particular less than 20 μm, and in particular less than 15 μm.

The above description of the cutting region 21 is disclosed in conjunction with all exemplary embodiments of this application.

In the exemplary embodiment according to FIG. 1, the meshes 24 each have four sides, which are each formed by two webs 22. In this case, the webs 22 have an S-shaped contour, wherein they are oriented substantially in the same direction on one side of the mesh 24. By contrast, the cells 23 according to FIG. 1 each have four sides, which are each formed by a single web 22.

In the case of both the cells 23 and the meshes 24, two webs 22 that are interconnected and belong to different sides of the cell 23 or mesh 24 have a different orientation.

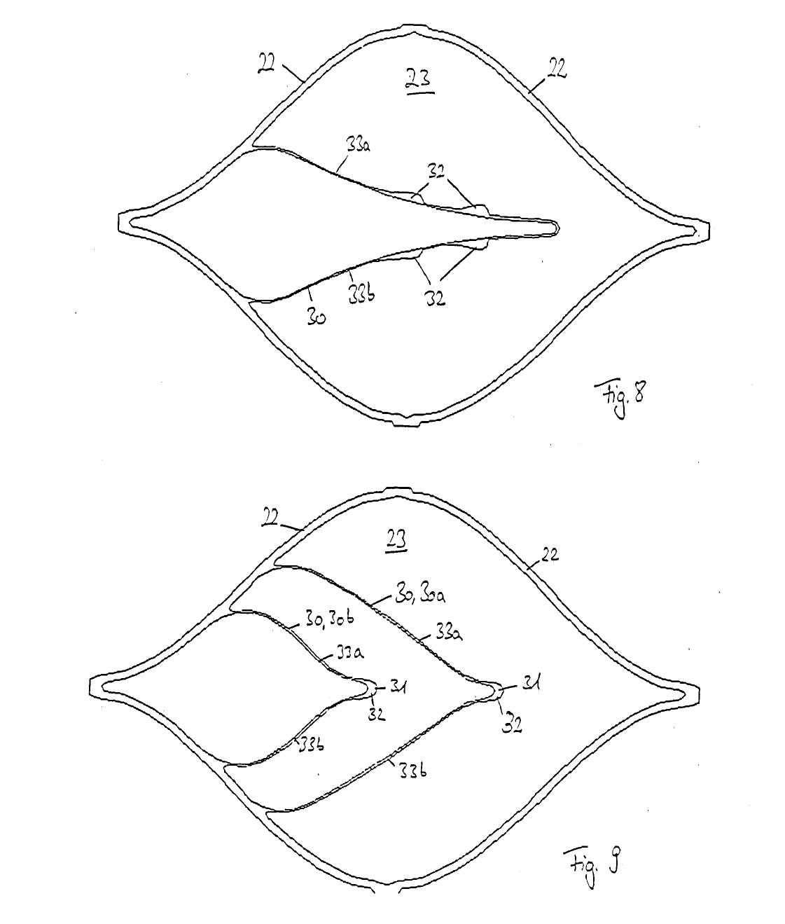

In the exemplary embodiment according to FIG. 1, a holding element 30 is also arranged in each of the meshes 24. The holding element 30 forms a tongue-shaped cell with a free tip, which projects into the mesh 24 (cell-in-cell or cell-in-mesh arrangement). It is also possible to arrange the holding element 30 in a cell 23 (see FIGS. 5 and 9). In particular, a first holding element 30a is arranged in a mesh 24 and a second holding element 30b is arranged in a further mesh 24. The holding elements 30, 30a, 30b are generally connected to the lattice structure 20, specifically to the webs 22 of the lattice structure 20. In particular, the holding elements 30, 30a, 30b are formed in one piece with the lattice structure 20. In the exemplary embodiment according to FIG. 1, the holding elements 30, 30a, 30b are each connected to two node points 25 of the lattice structure 20. In particular, the holding elements 30, 30a, 30b each have two spokes 33a, 33b, which are each connected to a node point 25. The spokes 33a, 33b form a V-shaped progression of the holding element 30. The holding element 30 further has a tip 31, in which the spokes 33a, 33b are interconnected.

The tip 31 forms a connection element between the lattice structure 20 and a guide wire 40 (not illustrated). To this end, the lattice structure 20 may have different geometrical forms. In the exemplary embodiment according to FIG. 1, the tip 31 of the first holding element 30a forms an eyelet 31a, in which the guide wire 40 can be arranged so as to be axially displaceable. The eyelet 31a basically has a contour that corresponds approximately to the contour of a three-quarter circle. By contrast, the tip 31 of the second holding element 30b forms a loop, of which the contour corresponds substantially to the contour of a semi-circle. In both cases, the tip 31 of the holding element 30 is formed in one piece with the spokes 33a, 33b of the holding element 30. The tip 31 may also be provided without connection to the guide wire and may thus be freely movable.

Compared to the cutting region 21 or the webs 22 of the lattice structure 20, the holding element 30 has a greater flexibility or resilience. During the expansion of the functional element 10, the holding element 30 is therefore radially deflected relatively easily. The holding element 30 extends radially inwardly in the expanded state of the functional element 10. The connection between the holding element 30 and the guide wire 40 ensures the radial deflection of the holding element 30 during the expansion of the functional element 10.

The radial deflection of the holding element 30 in the expanded state of the functional element 10 is illustrated in FIGS. 2a and 2b. In this case, the functional element 10 has a single holding element 30, which is substantially V-shaped and has a tip 31 formed as a loop 31b. The guide wire 40, which extends substantially longitudinally axially through the functional element 10, runs in the loop 31b. The holding element 30 may be connected to the guide wire 40 so as to be axially displaceable. In other words, the guide wire 40 is placed substantially loosely into the loop 31b. The tip 31 of the holding element 30 may thus slide over the guide wire 40. Alternatively, the tip 31 may be fixedly connected to the guide wire 40. An axially displaceable mounting of the tip 31 on the guide wire 40 is preferable, wherein the materials or surfaces of the guide wire 40 and of the holding element 30, in particular the tip 31, can be adapted in such a way that there is minimal friction between the tip 31 and the guide wire 40.

It is also possible for the functional element 10 to comprise a plurality of holding elements 30, which are connected in an axially displaceable manner to the guide wire 40, as illustrated in FIGS. 3a and 3b. In the exemplary embodiment according to FIGS. 3a and 3b, the holding elements 30 each have a V-shaped contour with a tip 31 formed as a loop 31b. Each loop 31b of the individual holding elements 30 loops around the guide wire 40. As illustrated in FIG. 3a, a plurality of holding elements 30 are arranged in succession in the longitudinal axial direction of the functional element 10, and in particular are arranged in succession in an aligned manner. The holding elements 30 are thus spaced in the longitudinal axial direction of the functional element 10. In addition, as illustrated in FIG. 3b, a plurality of holding elements 30 may be distributed over the circumference of the lattice structure 20 at the same cross-sectional height. In this case, two holding elements 30 may be arranged opposite one another in each case. The holding elements 30 may be arranged in a cross shape, such that the holding elements 30 are each distributed over the circumference offset by 90 degrees. As a result, four holding elements 30 are each arranged at a cross-sectional height. Another number of holding elements at a cross-sectional height, which are then distributed over the circumference, offset by a different circumference, is possible. For example, 3 holding elements 30 or more than 4 holding elements 30 may be provided. Alternatively, the holding elements 3 may also be arranged in a helical or spiral manner. The holding elements 30 arranged in succession in the longitudinal axial direction may thus each be arranged offset by an angle about the longitudinal axis of the functional element 10, such that a spiral arrangement is formed on the hole. Generally, the holding elements 30 may have different lengths so as to ensure that the guide wire 40 is arranged longitudinally axially, in particular in a straight line, in the functional element 10 in the expanded state of the functional element 10. This is true for all exemplary embodiments.

A lattice structure 20 with holding elements 30 that have different lengths is illustrated for example in FIG. 16. In this case, a first holding element 30a has a first length L1 and a second holding element 30b has a second length L2. The lengths L1, L2 are basically established along the longitudinal axis of the functional element 10 when the holding elements 30 are aligned in the longitudinal axial direction. Generally, the holding elements 30 can be arranged longitudinally axially, that is to say along the longitudinal axis of the functional element 10, and/or in the circumferential direction of the functional element 10. The length of the holding element 30 is accordingly established in the circumferential direction of the functional element 10 when the holding element 30 is oriented in the circumferential direction. As can be seen clearly in FIG. 16, the spokes 33a, 33b of the first holding element 30a and of the second holding element 30b are each connected in the same circumferential plane of the functional element 10 or of the lattice structure 20 to the webs of the lattice structure 20. In particular, the holding elements 30a, 30b are each connected to node points 25 of the lattice structure 20, which are arranged in the same cross-sectional plane or the same circumferential line of the functional element 10. Due to the different lengths L1, L2 of the holding elements 30a, 30b, the tips 31 of the holding elements 30a, 30b are by contrast arranged in different cross-sectional planes or circumferential lines of the functional element 10. As a result, the tips 31 of the holding elements 30a, 30b are connected to the guide wire 40 or are arranged on the guide wire 40 offset axially in relation to one another in the expanded state of the functional element 10, as illustrated in FIG. 17a. FIG. 17b shows the arrangement of the holding elements 30a, 30b in a cross-sectional illustration. It can be seen that the holding elements 30a, 30b are arranged transversely axially opposite one another in the expanded state of the functional element 10. Due to the offset arrangement of the spokes 31 of the holding elements 30a, 30b, it is ensured that the guide wire 40 is aligned substantially axially identically to the functional element 10.

The operating principle of the medical device is illustrated by way of example in FIGS. 4a to 4d. In FIGS. 4a to 4d, a blood vessel 65 is shown, in which a concretion 60 is arranged. The concretion 60 may comprise a thrombus, that is to say a blood clot, for example. The blood clot or the concretion 60 closes the blood vessel 65, so that a nutrient supply to tissue areas arranged downstream is interrupted. To remove the concretion 60, a catheter 50 is first guided to the treatment site, that is to say into the region of the concretion 60. The distal end of the catheter 50 may contact the thrombus or the concretion 60. The functional element 10 is displaced distally with the guide wire 40 as far as the concretion 60 through the hollow channel formed in the catheter 50. The functional element 10 is connected to the guide wire 40 during this process. The functional element 10 is compressed or is in the compressed state (FIG. 4a) within the catheter 50. In a next step, the catheter 50 with the functional element 10 and the guide wire 40 is displaced distally and, in so doing, penetrates the concretion 60 (FIG. 4b). The catheter 50 is then withdrawn in the proximal direction, whereby the outer restraint, which held the functional element 10 in the compressed state, is released. The functional element thus expands. Specifically, the functional element 10, in particular the lattice structure 20, has a radial force that causes the expansion of the lattice structure 290 or of the functional element 10. Here, the cutting region 21 of the lattice structure 20 penetrates the concretion 60. In other words, the webs 22 of the cutting region 21 of the lattice structure 20 cut into the concretion 60. In so doing, the functional element 10 widens in the radial direction. The concretion 60 is thus penetrated in the radial direction by the webs 22 or the cutting region 21. The holding elements 30 connected to the guide wire are deflected inwardly during the expansion of the functional element in the radial direction and thus form an anchoring for the concretion 60 (FIG. 4c). As soon as the functional element 10 is completely expanded, the concretion 60 can be removed from the blood vessel 65 by a proximal movement of the guide wire 40, which is connected fixedly to the functional element 10 (FIG. 4d).

When completely expanded, the functional element advantageously has a cross-sectional diameter that is smaller than the inner diameter of the blood vessel 65. Damage to the vessel wall of the blood vessel 65 caused by the functional element 10 is thus prevented, in particular when the concretion 60 is removed by withdrawing the functional element 10.

The connection between the holding elements 30 and the guide wire 40 ensures that the holding elements 30 span the lumen of the functional element 10 from the lattice structure 20, to which the holding elements 30 are connected, to the center line of the lattice structure 20. The functional element 10 thus has a large effective cross section for the anchoring of the functional element 10 to the concretion.

It is also possible to expand the functional element to a diameter that is smaller than the vessel diameter so as to maintain a distance from the vessel wall during removal of the functional element, so that said vessel wall is prevented from becoming damaged during the treatment process. In this regard, the radial force meets two conflicting requirements, namely applying a sufficient cutting force and also maintaining a spacing between the lattice structure and the vessel wall.

Although the possibility of gentle treatment by avoiding contact with the vessel wall is an advantage of the thrombectomy device, it is not ruled out that the lattice structure, for example for the treatment of concretions that can only be detached with difficulty, may be designed such that, in the expanded state, it comes into contact with the vessel wall or even exerts a radial force on the vessel wall in such a way that the vessel wall is expanded. The force applied to the concretion thus increases.

The lattice structure may have different diameters in the longitudinal direction, for example for the treatment of vessels of decreasing diameter. In this case, one region of the lattice structure may be smaller than the diameter of the vessel and another region may be the same size as, or larger than, the diameter of the vessel. The lattice structure may be designed such that the diameter of the lattice structure is smaller distally than it is proximally. The diameter thus decreases in the distal direction. The lattice structure is thus prevented from damaging a vessel of distally decreasing diameter or distal vessels of smaller diameter. On the other hand, the proximally enlarged diameter of the lattice structure affords improved anchoring of the lattice structure in the concretion in the region of the vessel of larger diameter.

It is also possible for the diameter of the lattice structure to be smaller proximally than it is distally. The diameter is thus larger in the distal direction. A greater radial force is thus exerted onto the vessel wall in the distal vessel region. The distal region of the lattice structure thus functions as a filter, which prevents detaching particles from being swept away and closing distally arranged vessel portions.

The diameter of the larger region of the lattice structure (distal or proximal) may be, for example, at least 20%, in particular at least 40%, in particular at least 60%, in particular at least 80%, and in particular at least 100%, larger than the diameter of the smaller region of the lattice structure.

For all exemplary embodiments, it is true that the holding element 30 preferably has a lower mechanical resistance or a greater flexibility than the webs 22 of the cutting region 21. For example, this may be achieved in that the width of the spokes 33a, 33b is smaller than the width of the webs 22. The spoke width S1 and the web width S2 is illustrated by way of example in FIG. 5. It can be clearly seen that the spoke width S1 is smaller than the web width S2. The width of the spokes 33a, 33b, that is to say the spoke width S1, may be between 10 μm and 50 μm. By contrast, the width of the webs 22, that is to say the web width S2, may be at least 20 μm and at most 100 μm.

It can also be seen in FIG. 5 that the holding element 30 is arranged within a cell 23 of the lattice structure. The spokes 33a, 33b are in this case each connected directly to a web 22 of the lattice structure 20. The shape of the holding element 30 corresponds, for the rest, to the shape of the second holding element 30 according to FIG. 1, wherein the tip 31 forms a loop 31b.

The functional element 10 generally has a cylindrical basic shape, both in the expanded state and in the compressed state. The rotationally symmetrical lattice structure 20 is thus discernible both in the expanded state and in the compressed state.

Upon expansion of the functional element 10 within the concretion 60, different degrees of expansion of the functional element 10 are reached due to the different mechanical properties or flexibilities or resiliences of the holding element 30 with respect to the webs 22 of the cutting region 21. The different mechanical properties or different mechanical rigidities lead to different local radial forces of the functional element 10. Specifically, the cutting region 21 has a greater radial force or expansion force than the holding element 30. Since the magnitude of the radial force determines the penetration potential of the functional element during expansion within the concretion 60, the webs 22 of the cutting region 21 cut into the concretion material, whereas the holding element 30 is deflected radially inwardly. The holding element 30 is thus deflected not only by the connection to the guide wire 40, but also by the external pressure of the concretion 60 on the holding element 30.

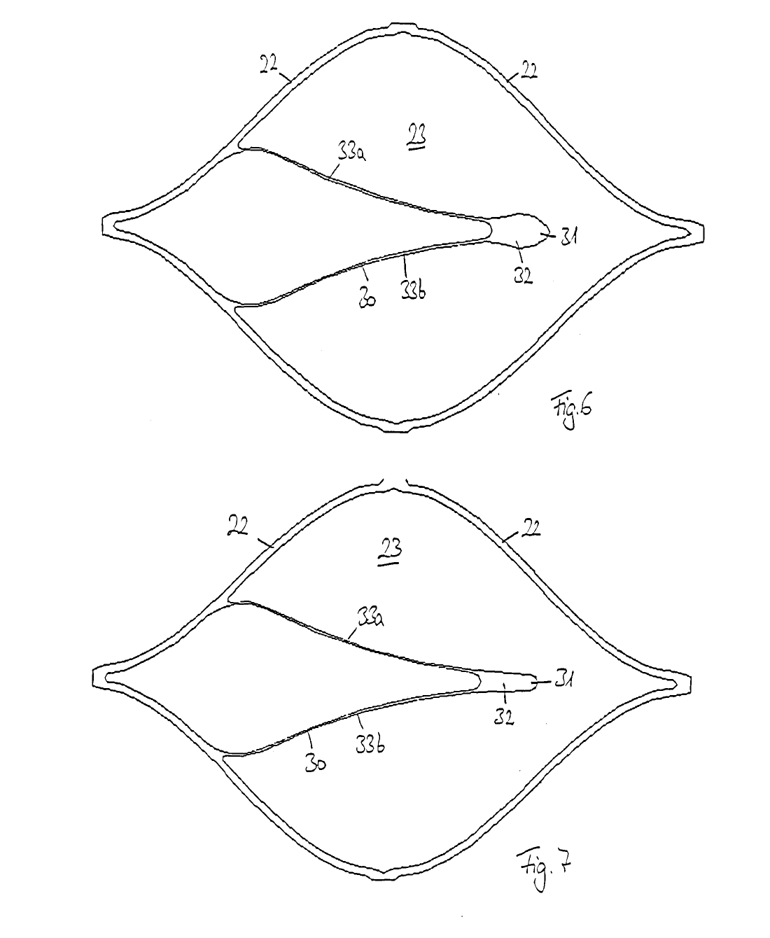

This effect can be used advantageously from a structural point of view so as to assist the radial deflection of the holding element 30. To this end, a contact area between the holding element 10 and the concretion 60 is to be enlarged in each of the exemplary embodiments according to FIGS. 6 to 8. In the exemplary embodiment according to FIG. 6, the tip of the holding element 30 has an enlarged contact area 32, for example. In particular, the tip 31 of the holding element 30 is spoon-like. A similarly formed contact area 32 of the tip 31 is illustrated in FIG. 7. The exemplary embodiments according to FIGS. 6 and 7 differ in that the contact area 32 according to FIG. 6 bulges convexly laterally so as to form a spoon-like contour. By contrast, the contact area 32 according to FIG. 7 has straight side edges, so that a spatula-like contour is instead formed.

A further variant for increasing the contact area between the holding element 30 and the concretion 60 is illustrated in FIG. 8. In this case, the spokes 33a, 33b each have at least one wing-like widening, which forms a contact area 32. Specifically, two wing-like widenings are provided on each of the spokes 33a, 33b in the exemplary embodiment according to FIG. 8. The wing-like widenings or contact areas 32 are preferably arranged at an outer contour of the V-shaped holding element.

A further exemplary embodiment is illustrated in FIG. 9, wherein the tips 31 of the holding elements 30 each have an enlarged contact area 32. Specifically, more than one holding element 30, in particular two holding elements 30, is/are arranged in a cell 23 in the exemplary embodiment according to FIG. 9. The holding elements 30 each have first and second spokes 33a, 33b. In this case, the first spokes 33a are connected to a first web 22a and the second spokes 33b are connected to a second web 22b. In this case, the holding elements are arranged in such a way that the tips 31 are arranged in succession in the longitudinal axial direction of the functional element 10 or the lattice structure 20. It is possible for all holding elements 30 of a cell 23 to be connectable to the guide wire 40. Alternatively, one holding element 30, in particular the first holding element 30a, which is larger than the second holding element 30b, may be connected to the guide wire 40, and the other holding element 30, in particular the second holding element 30b, may be arranged freely. In the case of the free holding element 30, in particular the second holding element 30b, the radially inwardly directed deflection during the expansion of the functional element 10 is not triggered via the guide wire 40, but substantially automatically by the cooperation between the free holding element 30 and the concretion 60.

The lattice structure 20 of the functional element 10 may generally have holding elements 30, which are connected to the guide wire 40 and comprise further, free holding elements 30, which are arranged loosely.

The mechanical properties or mechanical rigidities, which differ in some regions, of the lattice structure 20 may be achieved by different geometrical designs of individual elements of the lattice structure 20. Different widths of the webs 22 or the spokes 33a, 33b of the holding element 30 may advantageously be provided. Different widths of different elements of the lattice structure 20 are illustrated in FIG. 10. For example, the spokes 33a, 33b of the holding element 30 have a spoke width S3. The lattice structure 20 further has a cutting region 21, which is formed from a plurality of first webs 22a having a first web width S5 and a plurality of second webs 22b having a second web width S6. The spoke width S3 is preferably smaller than the first web width S5. The first web width S5 is preferably smaller than the second web width S6. The second webs 22 thus form part of the cutting region 21, which has a greater penetration force than the part of the cutting region 21 formed by the first webs 22a. The second webs 22b preferably delimit cells 23, which form an axial portion or a cell ring 26 of the lattice structure 20. The spoke width S3 of the spokes 33a, 33b of the holding element 30 is preferably at least 10 μm and/or at most 50 μm. A range from at least 20 μm to at most 100 μm is advantageous for the web widths S5, S6, wherein the first web width S5 is smaller than the second web width S6.

In the exemplary embodiment according to FIG. 10, the tip 31 of a first holding element 30 has an enlarged, in particular spatula-shaped, contact area 32. The contact area 32 or the tip 31 comprises a tip width S4, which is preferably at least 50 μm and/or at most 500 μm. A second holding element 30b likewise has a tip 31 having an enlarged contact area 32. The tip 31 forms a loop 31b. Furthermore, the second holding element 30b comprises a transition region 35, which is formed between each of the spokes 33a, 33b and the tip 31. The transition region is characterized by a widened contour of the spokes 33a, 33b. Specifically, the transition region 35 may have a transition width S7, which is at least 50 μm. The transition width S7 may be at most 200 μm.

The operating principle of the medical device is determined in particular by the geometry of the lattice structure 20, specifically by the size of the cells 23. For example, comparatively large cells 23, which are delimited by webs 22, have a large web cross section, an increased radial force and therefore less flexibility. Cells 23 with webs 22, which have a large web cross section, preferably form a cutting region 21, since cells 23 or webs 22 shaped in such a way have a high penetration potential. The webs 22 of the cutting region 21, which comprise a large web cross section, thus enable improved cutting into a concretion 60. Smaller cells 23, of which the webs 22 have a smaller web cross section, have a relatively low radial force and therefore greater flexibility. Cells 23 or webs 22 formed in such a way may therefore exert a filtering or retaining effect on the concretion 60 or detaching concretion particles.

A plurality of exemplary embodiments of the functional element 10 are illustrated in FIGS. 11a to 11d, wherein the functional element 10 comprises a lattice structure 20 having a plurality of holding elements 30. In the exemplary embodiments according to FIGS. 11a, 11b and 11d, the holding elements 30 are each arranged in meshes 24 of the lattice structure 20. By contrast, the holding elements 30 in the exemplary embodiment according to FIG. 11b are arranged in the cells 23 of the lattice structure 20.

The functional element 10 has two axial ends 11, in particular a proximal end 11a and a distal end 11b. The proximal end 11a may be fixedly connected to the guide wire 40. In particular, the functional element 10 according to FIG. 13a has a proximal end region A, which is fixedly connected to the guide wire 40 (not illustrated). The fixed connection between the proximal end region A or the proximal end 11a of the functional element 10 and the guide wire 40 may be produced for example by gluing, welding, soldering or crimping. The functional element 10 also comprises a distal end 11b or a distal end region B, which is formed by holding elements 30. The holding elements 30 each have a tip 31, which may be formed as a loop 31b, eyelet 31a or sliding sleeve 31c. In the exemplary embodiment according to figures 11a to 11d, the holding elements 30 at the distal end 11b of the functional element 10 each have a sliding sleeve 31c. The sliding sleeves 31c are connected to the guide wire so as to be longitudinally axially displaceable. Specifically, the guide wire 40 can be guided through the sliding sleeves 31c. To this end, the holding elements 30 at the distal end 11b of the functional element or in the distal end region B advantageously have different lengths, as illustrated in FIGS. 11a to 11d, so that the sliding sleeves 31c are arranged in a staggered manner on the guide wire 40 in the axial direction. A plurality of holding elements 30, which are displaceably connected to the guide wire 40, extend over the circumference of the functional element 10 at the distal end 11b of the functional element 10. A net-like closure of the functional element 10 or the lattice structure 20 is thus formed in the expanded state of the functional element 10 at the distal end 11b of the functional element 10 or in the distal end region B. The functional element 10 thus has a closed distal end region B. In addition, it is also possible for the tips 31 of the holding elements 30 at the distal end 11b of the functional element 10 to be adapted and designed for the receipt of X-ray markers.

In the exemplary embodiment according to FIG. 11a, the lattice structure 20 has a plurality of first holding elements 30a and a plurality of second holding elements 30b, wherein the first holding elements 30a have a greater length L1, L2 than the second holding elements 30b. In this case, the second holding elements 30b are each arranged in a circumferential line or an axial portion of the lattice structure 20. This means that the second holding elements 30b are each arranged annularly in an axial portion of the functional element 10. By contrast, the first holding elements 30a are arranged offset from one another in the axial direction and in the circumferential direction. Specifically, the first holding elements 30a are arranged substantially helically or spirally in the lattice structure 20. The first holding elements 30a of the lattice structure 20 are preferably connected to the guide wire 40. The second holding elements 30b may likewise be connected to the guide wire 40. The second holding elements 30b preferably form freely or loosely arranged holding elements 30 in the exemplary embodiment according to FIG. 11a.

The exemplary embodiment according to FIG. 11b differs from the exemplary embodiment according to FIG. 11a by the arrangement of the holding elements 30. Specifically, a plurality of first holding elements 30a are arranged spirally or helically along the functional elements 10 in the exemplary embodiment according to FIG. 11b. Furthermore, a plurality of second holding elements 30b are provided, which are likewise arranged helically or spirally along the functional element 10, wherein a first holding element 30a and a second holding element 30b are in each case arranged in the same axial portion of the functional element 10. The first holding element 30a may have a greater length L1, L2 than the second holding element 30b. It is also possible for the first holding element 30a and the second holding element 30b to have the same length L1, L2. The first holding elements 30a and/or the second holding elements 30b may be connected to the guide wire 40.

In the exemplary embodiment according to FIG. 11c, a plurality of holding elements are provided, which have substantially the same length. Each holding element 30 is arranged in a cell 23 of the lattice structure 20 of the functional element 10. A holding element 30 is preferably arranged in each cell 23 of a cell ring 26 or an annular segment. On the whole, the functional element 10 has a plurality of first cell rings 26a and a plurality of second cell rings 26b, wherein holding elements 30 are arranged in the first cell rings 26a. The cells 23 of the second cell rings 26b are free or have no holding elements 30. Three second cell rings 26b are preferably arranged between two first cell rings 26a in each case. A different number of second cell rings 26b between two first cell rings 26a is possible.

A further exemplary embodiment is illustrated in FIG. 11d and shows a design of the lattice structure 20 similar to the exemplary embodiment according to FIG. 11a. The difference from the exemplary embodiment according to FIG. 11a lies in the fact that the holding elements 30, that is to say the first holding elements 30a and second holding elements 30b, have the same length L1, L2 in the exemplary embodiment according to FIG. 11d.

With regard to the axially displaceable connection between the functional element 10 and the guide wire 40, an alternative connection option is illustrated in FIGS. 12a to 13. The tip 31 may comprise a sliding sleeve 31c, either alternatively or additionally to an eyelet 31a or a loop 31d. The sliding sleeve 31c may be connected to the tip 31 or generally to the holding element 30 by welding, gluing, soldering or crimping. The sliding sleeve 31c may be arranged axially displaceably on the guide wire 40. It is possible for a plurality of holding elements 30 to be connected to a sliding sleeve 31c, as illustrated in FIGS. 12a and 12b. Alternatively, each holding element 30 may be assigned a single sliding sleeve 31c, as shown in FIG. 13. The sleeves 31c of different holding elements 30 are preferably arranged in a staggered manner in the axial direction of the guide wire 40. Generally, the sliding sleeve 31c may comprise a metal, a plastic and/or a radiopaque material.

It is shown in FIGS. 16a to 16c that the holding elements 30 can be oriented in different directions. In the exemplary embodiment according to FIG. 16a, a plurality of holding elements 30 are provided for example, which are arranged in meshes 24 of the lattice structure 20. The meshes 24 or the holding elements 30 extend spirally along the functional element 10 or the lattice structure 20. The holding elements 30 have the same directional orientation. Specifically, the holding elements 30 in the exemplary embodiment according to FIG. 16a are oriented in the distal direction. This means that the tip 31 of the holding element 30 points in the direction of a distal end 11b of the functional element 10. By contrast, in the exemplary embodiment according to FIG. 16b, the holding elements 30 are oriented in the proximal direction. The tips 31 of the holding elements 30 according to FIG. 16b thus point in the direction of a proximal end 11a of the functional element 10. A combination of proximally oriented holding elements 30 and distally oriented holding elements 30 is likewise possible, as illustrated in FIG. 16c.

FIGS. 17a to 19b show exemplary embodiments of the medical device, wherein the holding elements 30 are each arranged in meshes 24 of the lattice structure 20. FIGS. 17a to 19b illustrate the different variants of the arrangement of the holding elements 30. For example, it is illustrated in FIG. 17a that a plurality of holding elements 30 can be arranged offset both in the axial direction and in the circumferential direction of the lattice structure 20. In this case, a single holding element 30 is arranged in each axial portion of the lattice structure 20 or the functional element 10. The holding elements offset from one another in the circumferential direction are arranged diametrically opposed in the cross-sectional view according to FIG. 17b. The V-shaped holding elements thus form an X-shaped structure in the cross-sectional view according to FIG. 17b. The holding elements 30 according to FIGS. 17a to 19b are each connected to the guide wire 40 (not illustrated).

In the exemplary embodiment according to FIGS. 18a and 18b, three holding elements 30, or generally a plurality of holding elements 30, are in each case arranged in an axial portion of the functional element 10. The holding elements 30 are aligned both in the axial direction and in the circumferential direction of the functional element 10. In this case, the holding elements 30 have the same directional orientation. It can be seen in the cross-sectional view according to FIG. 18b that the holding elements 30 are each arranged at an angle of approximately 120 degrees over the circumference of the lattice structure 20. A star-shaped contour of the holding elements 30 is thus produced in the cross-sectional view.

The exemplary embodiment according to FIGS. 19a and 19b has a similar structure. Specifically, a star-shaped contour of the holding elements 30 can likewise be seen in the cross-sectional view according to FIG. 19b. In contrast to the exemplary embodiment according to FIGS. 18a and 18b, a plurality of first holding elements 30a and a plurality of second holding elements 30b are provided in the exemplary embodiment according to FIGS. 21a, 21b, wherein the second holding elements 30b have a different directional orientation compared to the first holding elements 30a. Specifically, the second holding elements 30b are arranged in the opposite direction or in a mirror-inverted manner with respect to the first holding elements 30a.

FIGS. 20a to 20d show exemplary embodiments of the medical device, wherein the functional element 10 has a plurality of holding elements 30, which have different directional orientations. In the exemplary embodiment according to FIG. 20a, two first holding elements 30a are provided for example, which are offset in the axial direction and have the same directional orientation. Two second holding elements 30b are likewise arranged axially offset from one another and have the same directional orientation. The directional orientation of the second holding elements 30b is reversed compared to the directional orientation of the first holding elements 30a. This means that the tips 31 of the first holding elements 30a and of the second holding elements 30b are directed toward one another. Specifically, the first holding elements 30a are oriented in the distal direction and the second holding elements 30b are oriented in the proximal direction.

The expansion behavior of the functional element 10 can be influenced by a suitable arrangement of the holding elements 30, in particular by suitable setting of the axial offset spacing between the holding elements 30, as illustrated in FIG. 20b. In this case, the second holding elements 30b are axially offset from one another in such a way that, with a predefined degree of expansion of the functional element 10, the tips 31 of the second holding elements 30b rest against one another and counteract further expansion of the functional element. The expansion of the functional element 10 is thus limited in the region of the second holding elements 30b. A substantially frustum-shaped contour of the rotationally symmetrical lattice structure 20 is thus produced in the expanded state of the functional element 10.

In the exemplary embodiment according to FIG. 20b, the second holding elements 30b are arranged at the proximal distal end 11a of the functional element 10, such that the lattice structure 20 tapers in the proximal direction in the expanded state of the functional element 10. Alternatively, the axial spacing between the first holding elements 30a can be set such that the lattice structure 20 tapers in the distal direction in the expanded state of the functional element 10, as illustrated in FIG. 20d. In addition, it is possible for both the axial spacing between the first holding elements 30a and the axial spacing between the second holding elements 30b to be set in such a way that the tips 31 of the holding elements contact with a predefined degree of expansion of the functional element 10. For example, a cylindrical lattice structure in the expanded state of the functional element 10 can thus be provided, of which the maximum expansion diameter is limited. The delimitation of the expansion diameter, both over the entire lattice structure 10 and in axial portions of the lattice structure 10, has the advantage that contact between the lattice structure 20 or the functional element 10 and the vessel wall of a hollow organ of the body is prevented.

The exemplary embodiment according to FIG. 20c corresponds substantially to the exemplary embodiment according to FIG. 20a, wherein the orientation of the first holding elements 30a and of the second holding elements 30b is reversed in each case. This means that the first holding elements 30a according to FIG. 20c are oriented in the distal direction and the second holding elements 30b are oriented in the proximal direction. The holding elements 30a, 30b according to FIG. 20b have the same orientation.

It can also be clearly seen in FIGS. 20a to 20d that the cutting region 21, which is formed by the webs 22 of the lattice structure 20, penetrates the concretion or cuts into the concretion 60 during use. By contrast, the holding elements 30 are deflected radially inwardly due to the connection to the guide wire 40 and thus form an anchoring or fixing for the concretion 60.

The delimitation of the expansion of the functional element 10 may advantageously be assisted by the arrangement of at least one delimitation element 41 on the guide wire 40. Two sleeves 41b, which are arranged on the guide wire 40 and each form a delimitation element 41, are illustrated in FIGS. 21a and 21b. The sleeves 41b are fixedly connected to the guide wire 40. The orientation and arrangement of the holding elements 30 in the exemplary embodiment according to FIGS. 21a and 21b corresponds substantially to the orientation and arrangement of the holding elements 30 according to the exemplary embodiment illustrated in FIGS. 21a and 21b. The holding elements 30 are axially displaceably connected to the guide wire 40. This means that the holding elements 30 can slide along the guide wire 40. The sliding movement is delimited by the sleeves 41b or generally by the delimitation elements 41, as illustrated in FIG. 21b. Specifically, FIG. 21a shows the partly expanded state of the functional element 10, wherein the holding elements 30 can slide freely on the guide wire 40. The fully expanded state of the functional element 10 is illustrated in FIG. 21b, wherein the sliding movement of a first holding element 30a and of a second holding element 30b is stopped by the sleeves 41b in each case. The sleeve 41b thus forms a stop for the holding element 30.

As illustrated in FIGS. 22a and 22b, a groove 41a may be provided as a delimitation element 41, either alternatively or additionally to the sleeve 41b. In this case, FIG. 22a shows the partly expanded state of the functional element 10, wherein the holding elements 30 can slide freely on the guide wire 40. The holding elements 30 have a tip 31, which is formed as a loop 31b or eyelet 31a. With continued expansion of the functional element 10, a first holding element 30a and a second holding element 30b each slide into a respective groove 41a. The eyelet 31a or loop 31b of the tip 31 of the holding element 30 thus latches into the groove 41a (FIG. 22b). The expansion of the functional element 10 is thus stopped.

During the conversion from the radially compressed state into the radially expanded state of the functional element, not only is the cross-sectional diameter of the rotationally symmetrical lattice structure 20 increased, but the axial length of the lattice structure 20 is simultaneously shortened. Due to the axially displaceable connection of the holding elements 30 to the guide wire 40, the holding elements 30 can follow the shortening of the lattice structure 20 during the expansion of the functional element 10. However, it is also possible for a holding element 30 to be provided, which is connected fixedly to the guide wire 40, as illustrated in FIGS. 23a and 23b. In this case, the holding element 30 preferably has resilient properties, which compensate for the shortening of the lattice structure 20 during the expansion of the functional element 10 or lattice structure 20. Specifically, the length L1, L2 of the holding element may vary during the expansion of the functional element 10. In particular, the holding element 30, which is fixedly connected to the guide wire 40, has a greater length L1, L2 in the compressed state of the functional element 10 (FIG. 23a) than in the expanded state of the functional element 10 (FIG. 23b).

So as to compensate for the change in length of the lattice structure 20 or the functional element 10 during the expansion process, the guide wire 40 may also comprise a flexible element 42. A construction of this type is illustrated in FIGS. 24a and 24b, wherein the flexible element 42 is formed as a spring 42a. For reasons of clarity, the holding elements 30, which are fixedly connected to the guide wire 40, are not shown in the exemplary embodiment according to FIGS. 24a and 24b. In the compressed state, the functional element 10 has a length L3, which is greater than the length L4 of the functional element 10 in the expanded state. The spring 42a is therefore stretched in the compressed state of the functional element 10. The spring 42a is part of the guide wire 40. The spring 42a or generally the flexible element 42 can be formed in one piece with the guide wire 40. Alternatively, the flexible element 42 or the spring 42a may form a separate component, which is connected to the guide wire 40 by welding, gluing, soldering or by crimping. The flexible element 42 or the spring 42a is preferably adapted in such a way that a bias is produced in the compressed state of the functional element. The bias acting in the axial direction of the functional element may effectively assist the expansion of the functional element 10. In particular, the spring 42a may actively contract the functional element 10 as soon as the functional element 10 is released from the catheter 50. In this case, the cross-sectional diameter of the functional element 10 simultaneously increases such that the spring 42a contributes actively to the expansion of the functional element 10.

It is true for all exemplary embodiments that the lattice structure 20 in general, and specifically the webs 22, and/or the holding elements 30 may have an increased surface roughness or generally a surface structuring. A biologically active surface, which promotes a biochemical connection between the concretion 60 or a thrombus and the functional element 10, is thus provided. The webs 22 or generally the lattice structure 20 may also be coated with substances that have an adhesive effect or are biologically active so as to reinforce the connection between the functional element 10 and the concretion 60 or a thrombus. For example, thrombogenic substances may be attached to the lattice structure 20.

So as to set the mechanical rigidity or mechanical properties of the holding elements 30, angular ratios between the webs 22 and the spokes 33a, 33b of the holding element 30 can also be set at points of the width of the spokes 33a, 33b or webs 22. Specifically, the spokes 33a, 33b of the holding elements 30 may have a different angle in relation to the longitudinal axis of the functional element 10 compared to the webs 22 of the cutting region 21 of the lattice structure 20. For example, the angles of the spokes 33a, 33b of the holding element 30 may be set in such a way that a plurality of holding elements 30 overlap with the expansion of the functional element 10, so that the material of the concretion 60 or thrombus material is practically crushed between two holding elements 30.

In accordance with the exemplary embodiments according to FIGS. 25a to 29, the lattice structure 20 comprises at least one catcher element 34, which protrudes radially outwardly beyond the wall of the lattice structure 20 in the expanded state of the lattice structure 20. The catching effect of the functional element 10 is improved by the catcher element 34. As described above, the lattice structure is not expanded completely to the vessel diameter during use. Rather, the diameter of the functional element 10 is smaller than the vessel diameter, so that the lattice structure 20 does not contact the vessel wall. In the ideal case, an annular gap is thus formed between the vessel wall and the lattice structure 20 of the functional element and is filled at least in part by the concretion 60 to be removed. Since the catcher element 34 protrudes radially outwardly beyond the lattice structure 20, the catcher element 34 covers the gap between the lattice structure 20 and the vessel wall and entrains the parts of the concretion located in the gap when the functional element 10 is withdrawn. The efficacy of the functional element is thus improved, since the effective cross section of the functional element 10 is enlarged, more specifically radially outwardly, as is the case with the inwardly directed holding elements 30. In this case, the entire lattice structure 20 does not lie against the vessel wall, but merely the catcher element 34 or a plurality of individual catcher elements 34. As will be discussed hereinafter in detail, the catcher elements 34 may be formed atraumatically so that the concretion can be removed more gently compared to the prior art, even if individual parts of the functional element 10 contact the vessel wall.

The design of the functional element 34 will be explained in greater detail on the basis of the following exemplary embodiments. As can be seen in FIG. 25a, the functional element 10 has at least one catcher element 34, in particular a plurality of catcher elements 34, which protrude(s) radially outwardly beyond the lattice structure 20 in the expanded state of the functional element 20. It may be sufficient to provide a single catcher element 34. A plurality of catcher elements 34 are expediently arranged over the circumference of the lattice structure 20, for example two catcher elements 34 at the same cross-sectional height. The catcher elements may be arranged diametrically opposed over the circumference of the lattice structure 20, as illustrated by way of example in FIG. 25b. It is also possible to provide more than 2, for example 3, 4, 5 or more, catcher elements 34 distributed over the circumference at the same cross-sectional height. A different number of catcher elements at the same cross-sectional height is likewise possible. Furthermore, a plurality of catcher elements 34 can be arranged spaced from one another in the longitudinal direction of the functional element. This arrangement can be combined with the arrangement of the catcher elements 34 distributed over the circumference. The catcher elements 34 may be aligned in the longitudinal direction. It is also possible to arrange the catcher elements 34 so as to be spaced from one another in the longitudinal direction and offset from one another in the circumferential direction. In this case, a spiral-shaped distribution of the catcher elements 34 over the circumference may be provided. Other distribution patterns of the catcher elements 34 are possible.

The catcher elements 34 may be combined with the holding elements 30, which extend into the lumen of the functional element. To this end, the features of all exemplary embodiments, including the features disclosed in the claims, are disclosed together with the features of the exemplary embodiments concerning the catcher element 34, in particular the exemplary embodiments according to FIGS. 25a to 29. An example for the combination of the catcher element 34 or the catcher elements 34 with the holding elements 30 is illustrated in FIGS. 25a to 25c. For example, two holding elements 30 can be combined with two catcher elements 34, which are each arranged at the same cross-sectional height. This arrangement can be repeated in the longitudinal direction of the functional element, as illustrated in FIG. 25c. The arrangement of the catcher elements 34 and of the holding elements 30 is offset by 90 degrees at the same cross-sectional height (FIG. 25b). In the development according to FIG. 25c, the catcher elements 34 and the holding elements are arranged alternately in the circumferential direction, thus producing the arrangement shown in FIG. 25b with the cylindrical shape of the lattice structure 90. The catcher elements 34 and the holding elements 30 are connected at the same height per annular segment or per cell ring 26, as can be seen in FIGS. 25a and 25c. A spaced connection between the catcher elements 34 and the holding elements 30 as well as combinations of the different connection points are possible.

The shape of the catcher elements 34 corresponds to the shape of the holding elements 30. The difference between the catcher elements 34 and the holding elements 30 lies in the fact that the catcher elements 34 protrude outwardly beyond the wall of the lattice structure 20 in the expanded state, whereas the holding elements 30 project into the lumen of the functional element, that is to say protrude radially inwardly beyond the wall of the lattice structure 20. In this case, the end or the tip 31 of the holding element 30 or of some holding elements 30 spaced from the lattice structure 20 is connected to the guide wire 40, as illustrated in FIGS. 25a and 25b. The end of the catcher elements spaced from the lattice structure 20 is a free end 34a. In the expanded state, the free end 34a is arranged outside the lattice structure 20, so that there is a spacing between the free end 34a and the lattice structure 20.

As illustrated in FIG. 25c, the shape of the catcher elements 34 corresponds to the shape of the holding elements 30. Specifically, the catcher elements 34 are V-shaped. The catcher elements 34 have spokes 33a, 33b, similarly to the holding elements 30, said spokes converging at the free end 34a or the tip of the catcher elements 34, where they are connected. The spokes 33a, 33b are widened toward the lattice structure 20. The ends of the catcher element 34 opposite the free end 34a are connected to the lattice structure 20, in particular in the region of node points 25 of the lattice structure 20.

The above-described shape of the catcher elements 34 is likewise provided with the holding elements 30, with the exception of the freely arranged end 34a. It is also possible to form the catcher elements 34 and the holding elements 30 differently in terms of shape.

The free end 34a of the catcher element 34 is oriented in the distal direction. This means that the free end 34a is oriented toward the distal end of the functional element 10, that is to say away from the catheter. The opening of the V-shaped catcher element 34 points in the proximal direction, that is to say toward the catheter. The reference to the catheter in order to define the orientation of the catcher element 34 relates to the released state of the functional element, in which the functional element 10 is arranged distally from the catheter tip. The opening of the catcher element 34 in the proximal direction is important for the catching effect and also for the atraumatic movement of the catcher element 34. In the exemplary embodiment according to the FIGS. 25a to 25c, the catcher elements 34 and the holding elements are oriented in the same direction. It is also possible to orient the catcher elements 34 and the holding elements 30 in different directions, in such a way that the free end 34a of the catcher elements 34 is oriented in the distal direction and the end or the tip 31, connected to the guide wire 40, of the holding elements 30 is oriented in the proximal direction. It is also possible to combine catcher elements 34 having the free end 34a oriented in the distal direction with holding elements 30 that are oriented in different directions, that is to say in the proximal direction and in the distal direction. Reference is made in this regard to the exemplary embodiments explained above in conjunction with the holding elements.

The use of a functional element 10 with catcher element 34 is illustrated in FIGS. 26a to 26c. The functional element 10 according to FIG. 26a comprises two catcher elements 34 arranged diametrically over the circumference (in accordance with FIG. 25b). The holding elements 30 are arranged at different cross-sectional heights, in such a way that the catcher elements 34 are connected to the lattice structure 20 between the holding elements 30 in the longitudinal direction. This possible arrangement of the catcher elements 34 and of the holding elements 30 at different cross-sectional heights is disclosed generally and in conjunction with all other exemplary embodiments. The holding elements 30 are provided in an X-arrangement. The tips 31 of the holding elements 30 are in this case each oriented inwardly in the longitudinal direction. The guide wire 40 is fixedly connected to the proximal end of the functional element 10. The catheter is not illustrated in FIGS. 26a to 26c. The direction of withdrawal is indicated by an arrow. It is also illustrated in FIGS. 26a to 26c that the vessel diameter increases in the proximal direction.

In the treatment step according to FIG. 26a, the functional element 10 is expanded in the region of the concretion 60. The expanded diameter of the functional element 10 is smaller than the vessel diameter, and therefore a gap forms between the lattice structure 20 and the vessel wall. The cutting region of the lattice structure 20 penetrates the concretion 60 radially, as can be seen in FIG. 26a. Due to the effect of the concretion, the catcher element 34 is pressed against the lattice structure 20 immediately after the release of the functional element 10 in the state shown in FIG. 26a. If the functional element 10 is moved in the direction of the arrow (FIG. 26b), the concretion 60 is entrained due to the anchoring of the cutting region in the concretion 60. The holding elements extending into the lumen of the functional element 10 are used in this case for security in the event that the cutting region of the lattice structure 20 is not sufficiently anchored in the concretion 60.

It can be seen in FIG. 26b that the concretion 60 and the lattice structure 20 or cutting region thereof become disengaged with increasing vessel diameter. The efficacy of the cutting region of the functional element 10 therefore decreases. The catcher elements 34 follow the retreating concretion 60, such that the anchoring effect is then produced between the concretion 60 and the functional element 10, even if the lattice structure 20 or the cutting region of the lattice structure 20 and the concretion 60 are completely disengaged (FIG. 26c). The catcher elements 34 then take on the holding function, since the concretion is forced into the spokes 33a, 33b, arranged in a V-shape, of the catcher element 34 as a result of the withdrawal movement, where it is squeezed together due to the V-shape of the catcher element 34. Due to the catcher elements protruding radially outwardly beyond the lattice structure 20, these follow the expanding vessel wall, at least to a specific degree, and thus ensure that the connection between the concretion 60 and the functional element 10 is maintained. The connection between the concretion 60 and the catcher elements 34 may be sufficient to remove the concretion 60 completely from the vessel. The penetration of the catcher elements 34 into the concretion can be clearly seen in cross section in FIG. 26d.

To improve the resilient properties of the catcher elements 34 with the objective of minimizing the traumatization of the vessel, the catcher elements 34 are as long as possible and have as many webs or spokes 33a, 33b as possible. Specifically, the length of the spokes 33b, 34b of the catcher element 34 is at least half, in particular at least two thirds, in particular at least three quarters, the length of a cell 23 in the longitudinal direction of the functional element 10, in which the catcher element 34 is arranged. The above-described features are also disclosed in conjunction with the holding elements 30. To protect the vessel wall, the catcher elements 34 have web widenings 34b in the region of the free end 34a so as to distribute the introduction of force into the vessel wall over a large area so that said introduction of force is thus gentler. The widening 34b may be elongate for example, in particular leaf-shaped (FIG. 27a). The widening 34b may also be circular, in particular spoon-like (FIG. 27b). Other shapes of the widening 34b are possible. The widening 34b adjoins the free end 34a in the longitudinal direction and lies substantially over the central axis of the catcher element 34 between the spokes 33a, 33b arranged in a V-shaped manner.

It is also possible for the free end 34a of the catcher element 34 to be bent inwardly or curved inwardly, in such a way that the free end 34a points in the direction of the lattice structure 20 in the expanded state (FIGS. 28a and 28b). The free end 34a may be curved to different degrees in the direction of the lattice structure 20, as illustrated in FIGS. 28a and 28b. As a result, an outer edge of the free end 34a running transversely to the direction of entry is reliably distanced from the vessel wall and, in the event of movement of the catcher element, the vessel wall only slides relative to the catcher element 34 in the region of the curvature.

A combination of different measures that contribute to a reduction of the traumatization of the vessel wall is presented in FIG. 29. The individual features of the exemplary embodiment according to FIG. 29 are disclosed and claimed both in combination with one another and also individually. As can be seen in FIG. 29, the catcher element 34 is divided into three regions A, B, C. The proximal region A is characterized in that the spokes 33a, 33b or webs of the catcher element 34 are relatively thin, in particular thinner than the webs 22 forming the cell 23 of the lattice structure 20. The flexibility of the catcher element 10 is thus improved, so that it can lie effectively against the vessel wall without damaging said wall. The middle region B has a web widening 34b provided on each of the opposed spokes 33a, 33b. The middle region B is arranged distally to the region A. The middle region B is arranged close to the free end 34a of the catcher element 34, in such a way that the widening 34b of the two spokes 33a, 33b contacts the vessel wall with the successive increasing enlargement of the vessel diameter. The force exerted onto the vessel wall by the catcher element 34 is thus distributed over a relatively large area. The free end 34a forms the distal region C and is curved radially inwardly by means of a suitable heat treatment, which will be described in greater detail at another point, so as to reduce the traumatization.

The arrangement of the catcher elements 34 is possible in the cells 23 and/or in the meshes 24, wherein the catcher elements 34 in the meshes 24 may be longer accordingly due to the larger lattice opening. The length ratio (at least two thirds, three quarters) mentioned in conjunction with the cells is also disclosed in conjunction with the larger meshes 24. It is also possible for the catcher elements 34 in the meshes 24 to be shorter, for example if the functional element 10 in a vessel is used with a relatively small increase in the vessel diameter in the proximal direction.

With use of a shape-memory material, which has the property of returning practically completely or partially to its original shape in the starting state following a change in shape in the martensitic state by heating, the shape-memory effect can be utilized to manufacture the inwardly curved free end 34a. In this case, the end 34a is curved in the expanded state and is straight in the compressed state. The crimpability of the functional element 10 is thus improved and the diameter of the functional element 10 in the crimped state is reduced.

A mandrel 55 having a radially protruding pin 55a is provided for the production process (see FIG. 31) and has a substantially cylindrical shape. The pin 55a is fixed into the free end 34a or the tip of the catcher element 34 and is rotated in a clockwise direction, so that the free end 34a is curved in the direction of the center of the lumen or in the direction of the lattice structure 20 (see FIG. 30a). By stopping the mandrel and by subsequent heat treatment, the deformation remains permanent. In the low-temperature phase, the free end 34a can be reshaped so as to adopt its original shape, in such a way that the catcher element is flat on the whole and can be fitted into the lattice structure 20 (FIGS. 30b and 30d). The catcher element 34 is heated during use, wherein the component reassumes its original radially outwardly protruding shape curved in the region of the free end 34a.

For the sake of completeness, it is disclosed that configurations of the functional element 10 are possible in which the elements, that is to say the catcher and holding elements 34, 30, are bent only inwardly or outwardly, or both inwardly and outwardly. A functional element that has only outwardly deflected catcher elements 34 is thus also disclosed.

It also possible to design the cutting region of the lattice structure 20 such that the holding element 30, that is to say the inwardly deflectable element, is surrounded proximally and distally in the longitudinal direction as well as in the circumferential direction of the lattice structure by lattice cells or meshes of the cutting region, wherein these lattice cells or meshes are arranged in the same cylindrical lateral surface of the lattice structure 20. This means that a holding element is surrounded in all directions by lattice cells of the cutting region, whereby the lattice structure cuts into the thrombus around the holding element. Due to the radially outwardly directed relative movement between the concretion and the lattice structure 20, the holding element 30 is moved or deflected inwardly in the direction of the centre of the lumen by the concretion 60.

In this instance, it is not ruled out that the cells 23 or meshes 24 surrounding a holding element 30 in turn also contain holding elements 30, such that these cells 23 or meshes 24 carry out two functions, namely they fasten the holding elements 30 and also act as cutting cells or cutting meshes. Due to the arrangement of the cutting region in the same lateral surface, a uniform cutting effect of the functional element is achieved in the longitudinal direction and in the circumferential direction of the lattice structure 20. The cutting effect and the associated anchoring effect of the cells or meshes 24 forming the cutting region are thus improved since the cells 23 or meshes 24 are substantially diamond-shaped, so that the webs 22 of the cells 23 or the meshes 24 are arranged at an incline with respect to the longitudinal axis of the functional element. The webs are arranged in the range of more than 0 degrees and less than 90 degrees based on the longitudinal axis.

In the case of a functional element as described above having a corresponding cutting region, it is possible to dispense with the fixing of the holding elements 30 on the guide wire 40, since in this case the movement or deflection of the holding elements 30 inwardly is caused by the resistance of the concretion 60. The fixing by means of the guide wire 40 has the advantage that the deflection of the holding elements 30 is implemented in any case, that is to say independently of the shape of the respective concretion, thus increasing the reliability of the device. It is possible to combine the above-described cutting region with the exemplary embodiments in which the holding elements 30 are connected to the guide wire, in particular fixedly or detachably connected. It is also possible, with a functional element 10, to provide holding elements freely, that is to say with no connection to the guide wire, and to provide holding elements connected to the guide wire.

The functional element 10 is disclosed and claimed both together with a catheter in the form of an arrangement comprising a functional element and a catheter. The functional element is also disclosed and claimed individually, that is to say without the catheter.

The functional element 10 may further comprise a displacement element 36. The functional element 10 preferably has a plurality of displacement elements 36. The displacement element 36 has substantially the same shape as the catcher element 34 or the holding element 30. In other words, the displacement element 36 may have a V-shaped structure. The displacement element 36 may thus accordingly have two spokes arranged in a V-shaped manner, wherein the spokes converge in a tip. The displacement element 36 is connected to at least two webs 22 of the lattice structure 20.

The displacement element 36 differs from the catcher element 34 in particular by its function. In the case of the catcher element 34, a radial deflection is produced by the conversion from the compressed state into the expanded state of the lattice structure 20. A mechanism of this type is not provided in the case of the displacement element 36. Rather, the radially outwardly directed deflection of the displacement element 36 is provided exclusively by a curvature of the functional element 10 or the lattice structure 20 along the longitudinal axis. In this case, the tip of the displacement element 36 advantageously points in the distal direction.

The specific operating principle of the displacement elements 36 may be set by the geometrical dimensions of the displacement elements 36. To this end, the displacement elements advantageously have a spoke width S1, which is at most 60 μm, in particular at most 50 μm, in particular at most 40 μm, in particular at most μm, and in particular at most 20 μm. The displacement element 36 preferably projects into a cell 23, wherein the webs 22 delimiting the cell 23 have a web width S2, which is at most 70 μm, in particular at most 60 μm, in particular at most 50 μm, in particular at most 40 μm, and in particular at most 30 μm. The displacement elements 36 thus generally have a spoke width S1, which is less than or equal to the web width S2 of the webs 22 surrounding the displacement element 36. A ratio between the spoke width S1 of the displacement element 36 and the web width S2 of the surrounding webs 22 (S1:S2) is particularly preferably at most 1.0, in particular at most 0.9, in particular at most 0.8, in particular at most 0.7, in particular at most 0.6, and in particular at most 0.5.

The operating principle of the displacement element 36 or of the displacement elements 36 will be explained hereinafter on the basis of FIGS. 32 to 32c.

The functional element 10 is first positioned and expanded in the blood vessel 65 in the region of the concretion 60. In so doing, the cutting region 21 of the lattice structure 20 cuts into the concretion 60, so that a connection is produced between the concretion and the functional element 10 (FIG. 32a). To remove the concretion 60, the functional element 10 is then moved or drawn in the proximal direction. The functional element 10 is bent or curved as it passes through a vessel curvature 66. In particular, the functional element 10 is subject to a longitudinal curvature as it passes through the vessel curvature 66. In other words, the rotational axis of the lattice structure 20 is converted from a straight-line arrangement into a curved or bent arrangement. A cavity 67 is thus exposed on an outer side of the longitudinal curvature, that is to say on a side of the lattice structure 20 having a greater radius of curvature than the opposed side of the lattice structure 20.

The cavity 67 provides a passing space for the concretion 60, and therefore the concretion 60 may detach from the catcher element 10. The reliable removal of the concretion 60 from the blood vessel 65 is thus placed at risk.

The formation of the cavity 67 is promoted in particular since an axial lengthening of the lattice structure 20 is caused in the region of vessel curvatures 66 due to the drawing of the functional element 10. The axial lengthening or extension of the lattice structure 20 also causes a radial compression of the functional element 10 or the lattice structure 20. The functional element 10 therefore no longer fills the entire cross-sectional diameter of the blood vessel 65. Rather, the cavity 67 is formed in an outer region of the vessel curvature 66.

The formed cavity 67 provides space for the concretion 60, and therefore the concretion 60 may detach from the functional element 10. This is prevented by the displacement elements 36. Due to the longitudinal curvature of the lattice structure 20, which is caused by drawing the functional element 10 through the vessel curvature 66, the displacement elements 36 automatically displace radially outwardly. In this case, the displacement elements 36 may lift or displace radially outwardly from the plane of the wall of the lattice structure 20 to varying degrees. The deflection of the displacement elements 36 may be dependent on the local radius of curvature of the lattice structure 20. In other words, the displacement elements 36 may have the same displacement angle, based on the lattice structure 20, wherein the distance between the tips of the displacement elements 36 and the lattice structure varies by longitudinal radii of curvature of the lattice structure 20 that are different over portions. In this case, the displacement elements 36 project into the cavity 67. The displacement elements 36 thus form a catch or a fixing for the concretion 60 in the deflected state (FIG. 32b).

The displacement elements 36 may engage in the concretion 60 or hook into the concretion 60, so that the concretion 60 is also fixed at the functional element 10. By drawing the functional element 10 further in the proximal direction, the concretion 60 is thus guided through the vessel curvature 66 (FIG. 32c). The displacement elements 36 activated by the anatomical conditions in the blood vessel 65 thus result in a reliable removal of the concretion 60. In particular, the security against loss of the concretion 60 to be removed is increased by the displacement elements 36.

The displacement elements 36 are oriented in the distal direction. This means that the displacement elements 36 have tips that point in the distal direction. The risk of damage to the vessel wall is thus reduced. The displacement elements 36 are preferably adapted or dimensioned in such a way that they project into the cavity 67 of a vessel curvature 66 without contacting the vessel wall.

Preferred dimensions of the displacement element 36 are illustrated in FIGS. 33 and 34. In particular, FIGS. 33 and 34 show preferred geometrical ratios of the web lengths or cell lengths and of the extension of the displacement element 36. The dimensions of the cells 23 in which the displacement element 36 is arranged and the dimensions of the cells 23 that have no displacement elements 36 are also important for the function of the displacement elements 36.

The overall length of the cell 23 in which no displacement element 36 is arranged is referred to as the overall length L8 of the cell 23 without displacement element 36. The overall length of the cell in which a displacement element 36 is arranged is referred to as the overall length L9 of the cell 23 with a displacement element 36. As is clear from FIG. 33, the overall length L9 of the cell 23 with a displacement element 36 is a multiple of the overall length L8 of the cell 23 without a displacement element 36.

In principle, the cells 23 are delimited by webs 22. In this case, the displacement element 36 divides two webs of the cell 23 into axial portions. Specifically, the displacement element 36 is connected to a web 22 of the cell 23, wherein a node point 25 is formed. The webs 22 of adjacent cells 23 are further interconnected in node points 25. The node point 25 in which the displacement element 36 is coupled to the web 22 is also referred to as an attachment point 25a. The axial distance between the attachment point 25a and the next node point 25, which is arranged opposite the tip of the displacement element 36, is referred to as the axial attachment distance L10. In this case, the axial attachment distance L10 is determined along the longitudinal axis of the functional element 10.

The axial attachment distance L10 is determined in principle in the expanded state of the lattice structure 10. The axial attachment distance L10 therefore differs from an attachment length L5, which is measured along the web 22 between the next node point 25 arranged opposite the tip of the displacement element 36 and the attachment point 25a (FIG. 34).

The displacement element 36 further has an axial length L11, which corresponds to the axial extension between the attachment point 25a and the tip of the displacement element 36. The axial length L11 of the displacement element 36 is likewise established in the expanded state of the lattice structure along the longitudinal axis of the lattice structure 20. The axial length L11 of the displacement element 36 thus differs from a spoke length L7 of the displacement element 36. The spoke length L7 of the displacement element 36 corresponds to the direct distance between the attachment point 25a and the tip of the displacement element 36, as illustrated in FIG. 37. In other words, the spoke length L7 corresponds to the length of a spoke of the displacement element 36, wherein the straight-line distance between a distal end of the spoke, which transitions into the tip, and a proximal end of the spoke, which corresponds substantially to the attachment point 25a, is used as a basis. The measurement therefore is not taken along the spokes, which are curved over portions, but along a virtual line or path between the ends of the spokes. The spoke length L7 of the displacement element 36 thus corresponds to the shortest connection between the attachment point 25a and the distal end of the spoke or the tip of the displacement element 36. Similarly to all other relevant measurements, the spoke length L7 of the displacement element 36 is also established in the expanded state of the lattice structure 20.

The web 22, which comprises the attachment point 25a, has the attachment length L5, which corresponds to the distance between a node point 25 and the attachment point 25a, measured along the web 22. The web 22 also has a remaining web length L6. The remaining web length L6 corresponds to the shortest distance between the attachment point 25a and a node point 25, which is arranged adjacent to the displacement element 36 in the circumferential direction of the lattice structure 20. The attachment length L5 and the remaining web length L6 are illustrated clearly in FIG. 34.

The lattice structure 20 preferably has the following dimensional ratios with respect to the displacement elements 36:

The ratio between the overall length L9 of the cell 23 with a displacement element 36 to the overall length L8 of the cell 23 without a displacement element 36 (L9:L8) is preferably at most 3.1, in particular at most 3.0, in particular at most 2.9, in particular at most 2.5, in particular at most 2.4, in particular at most 2.0, in particular at most 1.9, and in particular at most 1.5. The ratio of the axial length L11 of the displacement element 36 to the axial attachment distance L10 (L11:L10) is preferably at most 3.3, in particular at most 3.0, in particular at most 2.5, in particular at most 2.4, in particular at most 1.9, in particular at most 1.5, and in particular at most 1.0. The ratio between the overall length L8 of the cell 23 without a displacement element 36 and the axial attachment distance L10 (L8:L10) is preferably at most 2.5, in particular at most 2.3, in particular at most 2.0, in particular at most 1.8, and in particular at most 1.5. The lattice structure 10 preferably has a dimensional ratio between the overall length L8 of the cell 23 without a displacement element 36 and the axial length L11 of the displacement element 36 (L8:L11) that is at most 2.0, in particular at most 1.8, in particular at most 1.5, in particular at most 1.2, in particular at most 1.0, in particular at most 0.8, and in particular at most 0.5. Corresponding dimensional ratios are illustrated schematically in FIG. 33.

The length ratios between the attachment length L11, remaining web length L6 and spoke length L7 of the displacement element 36 are illustrated schematically in FIG. 34. In this case, the ratio between the attachment length L5 and the remaining web length L6 (L5:L6) is advantageously at most 2.0, in particular at most 1.8, in particular at most 1.5, in particular at most 2.2, in particular at most 1.0, in particular at most 0.7, and in particular at most 0.5. The ratio between the spoke length L7 of the displacement element 36 and the remaining web length L6 (L7:L6) is preferably at most 2.5, in particular at most 2.2, in particular at most 2.0, in particular at most 1.8, in particular at most 1.5, in particular at most 1.3, and in particular at most 1.0.

FIG. 35 shows a preferred design of a functional element 10 of the medical device, wherein the meshes 24 with holding elements 30 are offset in a zigzagged manner in circumferential rows. Cutting regions 21 are provided in each case between two circumferential sides with meshes 24 and comprise the cells 23 without holding elements 30. The holding elements 30 are oriented in the distal direction.

In addition, two paddles (no reference sign) are illustrated in FIG. 35 and form a preferred shape for connection of the device or functional element 10 to the transport wire. The paddles may be arranged oppositely. The transport wire is welded to the paddles on either side in two separate steps or else in a single welding step.

The functional element 10 is preferably produced by laser cutting. In this case, a cylindrical semi-finished product is used as raw material, into which the lattice structure 20 is introduced by means of laser cutting. The lattice structure 20 may then be subjected to a heat treatment, so that the inwardly directed deflection of the holding elements 30 is already established during the production process.

For example, the raw material may comprise a nickel/titanium alloy or generally a pseudoelastic material, in particular a shape-memory material. Due to the heat treatment, the inwardly deflected orientation of the holding elements 30 is impressed onto the shape-memory material, so that the holding elements 30 deflect radially inwardly substantially automatically upon expansion of the functional element 10. The heat-treated holding elements may also be combined with the guide wire 40. This effect can be promoted however by the heat treatment during the production process. The heat treatment is additionally advantageous if free holding elements 30 are provided, which are not connected to the guide wire 40. Alternatively to shape-memory materials, the lattice structure 20 or generally the functional element 10 may also comprise high-grade steel.

The invention is particularly suitable for the removal of thrombi from blood vessels, in particular from cerebral vessel portions.

To summarize, the device according to the invention is based on the concept of providing the lattice structure 20 with a cutting region 21, which, upon expansion of the lattice structure, is adapted to penetrate the concretion 60 adhering to the vessel. The cutting region 21 is assigned a holding element 30, in particular in the form of a tongue or a tongue-shaped cell with a tip. The holding element 30 assists the anchoring effect of the cutting region 21. To this end, the tongue is connected flexibly to the lattice structure 20 in such a way that, when the cutting region 21 penetrates the concretion, the concretion presses against the tongue and deflects the tongue radially inwardly. The anchoring and therefore the resistance of the functional element during the removal of the concretion from the vessel is improved by the radially inwardly deflectable tongue together with the cutting region, which surrounds the tongue at least in part. The holding element 30 or the tongue may additionally or alternatively be designed such that it moves radially outwardly from the cylindrical lateral surface in the event of a longitudinal curvature of the functional element. The tongue may thus follow the concretion if the cutting region detaches from the concretion, for example during the movement of the functional element along a vessel curvature. To this end, the tongue is located on the outer radius of the curved functional element.

In the rest state with an expanded functional element, the holding element 30 or the tongue is arranged in the same cylindrical lateral surface as the cutting region. Specifically, the tip of the tongue is arranged in the same cylindrical lateral region or the same cylindrical lateral surface of the lattice structure.

The above-explained design and operating principle of the device is disclosed in conjunction with all features of the description or generally in conjunction with the invention, without imposing any restriction thereto.

The following devices are also disclosed and claimed within the scope of the application:
1. A medical device for removing concretions (60) from hollow organs of the body, with a functional element (10), which has a rotationally symmetrical lattice structure (20) and a means for holding a concretion in the lattice structure (20), and a catheter (50) for introducing the functional element (10) into the body and for removing it therefrom, the functional element (10) being convertible from a compressed state in the catheter (50) into an expanded state outside the catheter (50), in which the functional element (10) is arranged distally from the catheter (50), characterized in that
the lattice structure (20) has a cutting region (21) with webs (22), which are adapted in such a way that they at least partially radially penetrate the concretion (60) during the conversion of the functional element (10) from the compressed state into the expanded state, and the holding means comprises at least one holding element (30), which is connected to the lattice structure (20) and also to a guide wire (40) arranged in the catheter (50) in such a way that the holding element (30) is deflected radially inwardly in the expanded state of the functional element (10).
2. The medical device as claimed in number 1,
characterized in that
the holding element (30) is connected fixedly or axially longitudinally displaceably to the guide wire (40).
3. The medical device as claimed in number 1 or 2,
characterized in that
the holding element (30) has a tip (31), which is connected to the guide wire (40).
4. The medical device as claimed in number 3,
characterized in that
the tip (31) has an eyelet (31a) or a loop (31b) or a sliding sleeve (31c), through which the guide wire (40) is guided.
5. The medical device as claimed in number 3 or 4,
characterized in that
the tip (31) has an enlarged, in particular spoon-like, contact area (32).
6. The medical device as claimed in at least one of numbers 1 to 5.
characterized in that
the holding element (30) is substantially V-shaped and comprises two spokes (33a, 33b), which are each connected to at least one web (22) of the cutting region (21).
7. The medical device as claimed in number 6,
characterized in that
the spokes (33a, 33b) of the holding element (30) have a lower mechanical rigidity, in particular a smaller width and/or thickness, than the webs (22) of the cutting region (21).
8. The medical device as claimed in at least one of numbers 1 to 7,
characterized in that
the lattice structure (20) comprises at least one catcher element (34), which protrudes radially outwardly beyond the lattice structure (20) in the expanded state.
9. The medical device as claimed in at least one of numbers 1 to 8,
characterized in that
the holding element (30) extends in the longitudinal axial direction of the functional element (10) in the compressed state of the functional element (10).
10. The medical device as claimed in at least one of numbers 1 to 9,
characterized in that
a plurality of holding elements (30) are provided and have different lengths.
11. The medical device as claimed in at least one of numbers 1 to 10,
characterized in that
a plurality of holding elements (30) are provided, which are arranged adjacently in the circumferential direction at an axial end (11) of the functional element (10) and are connected to the guide wire (40) so as to form a net-like closure of the functional element (10).
12. The medical device as claimed in at least one of numbers 1 to 11,
characterized in that
the guide wire (40) comprises at least one delimitation element (41), which delimits an axial movement of the holding element (30), in particular the tip (31), in the expanded state of the functional element (10).
13. The medical device as claimed in number 12,
characterized in that
the delimitation element (41) comprises a groove (41a), which is formed in the guide wire (40), or a sleeve (41b), which is fixedly connected to the guide wire (40).
14. The medical device as claimed in at least one of numbers 1 to 13,
characterized in that
the guide wire (40) comprises a flexible element (42), in such a way that the guide wire (40) can be longitudinally axially lengthened.
15. The medical device as claimed in at least one of numbers 1 to 14,
characterized in that
the functional element (10) has at least one displacement element (36), which is arranged flush in the lattice structure (20) with a straight-line arrangement of the functional element (10) and is deflected radially outwardly with a longitudinally curved arrangement of the functional element (10).
16. A medical device for removing concretions (60) from hollow organs of the body, with a functional element (10), which has a rotationally symmetrical lattice structure (20) and a means for holding a concretion (60) in the lattice structure (20), and a catheter (50) for introducing the functional element (10) into the body and for removing it therefrom, the functional element (10) being convertible from a compressed state in the catheter (50) into an expanded state outside the catheter (50), in which the functional element (10) is arranged distally from the catheter (50), characterized in that
the lattice structure (20) has a cutting region (21) with webs (22), which, during use, at least partially radially penetrate the concretion (60) during the conversion of the functional element (10) from the compressed state into the expanded state, and the holding means comprises at least one holding element (30), which is surrounded proximally and distally in the longitudinal direction as well as in the circumferential direction of the lattice structure (20) by lattice cells (23) and/or meshes (24) of the cutting region, the lattice cells (23) and/or meshes (24) of the cutting region being arranged in the same cylindrical lateral surface of the lattice structure (20).

### LIST OF REFERENCE SIGNS

10 functional element
11 axial end
11a proximal end
11b distal end
20 lattice structure
21 cutting region
22 web
22a first web
22b second web
23 cell
24 mesh
25 node point
25a attachment point
26 cell ring
26a first cell ring
26b second cell ring
30 holding element
30a first holding element
30b second holding element
31 tip
31a eyelet
31b loop
31c sliding sleeve
32 contact area
33a first spoke
33b second spoke
34 catcher element
34a free end
34b web widening
35 transition region
36 displacement element
40 guide wire
41 delimitation element
41a groove
41b sleeve
42 flexible element
42a spring
50 catheter
55 mandrel
55a pin
60 concretion
65 blood vessel
66 vessel curvature
67 cavity
A proximal end region
B distal end region
L1 length of the first holding element 30a
L2 length of the second holding element 30b
L3 length of the functional element 10 in the compressed state
L4 length of the functional element 10 in the expanded state
L5 attachment length
L6 remaining web length
L7 spoke length of the displacement element 36
L8 total length of the cell 23 without displacement element 36
L9 total length of the cell 23 with displacement element 36
L10 axial attachment distance
L11 axial length of the displacement element 36
S1, S3 spoke width
S2 web width
S4 tip width
S5 first web width
S6 second web width
S7 transition width

1. A medical device for removing concretions from hollow organs of the body, with a functional element, which has a rotationally symmetrical lattice structure and a means for holding a concretion in the lattice structure, and a catheter for introducing the functional element into the body and for removing it therefrom, the functional element being convertible from a compressed state in the catheter into an expanded state outside the catheter, in which the functional element is arranged distally from the catheter,
wherein
the lattice structure has a cutting region with webs, which, during use, at least partially radially penetrate the concretion during the conversion of the functional element from the compressed state into the expanded state, and the holding means comprises at least one holding element, which is surrounded proximally and distally in the longitudinal direction as well as in the circumferential direction of the lattice structure by lattice cells and/or meshes of the cutting region, the lattice cells and/or meshes of the cutting region being arranged in the same cylindrical lateral surface of the lattice structure.

2. The device as claimed in claim 1,
wherein
the holding element forms a tongue-shaped cell with a free tip, which is arranged in a diamond-shaped lattice cell and/or mesh of the lattice structure.

3. The device as claimed in claim 1, wherein
the lattice cell or mesh in which the holding element is arranged is more flexible, at least in some regions and in particular completely, than the lattice cells and/or meshes of the cutting region that surround the holding element.

4. The device as claimed in
claim 1, wherein
the lattice cell or mesh in which the holding element is arranged is smaller than, the same size as, or larger than the surrounding lattice cells or meshes -of the cutting region.

5. The device as claimed in
claim 1, wherein
the ratio of the web length of the lattice cell or mesh in which the holding element is arranged to the web length of the surrounding lattice cells or meshes of the cutting region is at least 110%, in particular at least 120%, at least 130%, at least 140%, at least 150%, at least 175%, or at least 200%.

6. The device as claimed in
claim 1, wherein
the holding element has webs, of which the width is at most 150%, in particular at most 120%, at most 110%, at most 90%, at most 80%, at most 70%, at most 60%, at most 50%, or at most 40%, of the web width of the surrounding lattice cells or meshes of the cutting region.

7. The device as claimed in
claim 1, wherein
the ratio of the width of the webs of a lattice cell or mesh in which the holding element is arranged to the web width of the surrounding lattice cells or meshes is at most 200%, in particular at most 150%, at most 120%, at most 90%, at most 80%, at most 70%, at most 60%, or at most 50%.

8. The device as claimed in
claim 1, wherein
the holding element is substantially V-shaped and comprises two spokes, which are each connected to at least one web of the cutting region.

9. The device as claimed in claim 8,
wherein
the spokes of the holding element have a lower mechanical rigidity, in particular a smaller width and/or thickness, than the webs of the cutting region.

10. The device as claimed in
claim 1, wherein
the lattice structure comprises at least one catcher element, which protrudes radially outwardly beyond the lattice structure in the expanded state.

11. The device as claimed in
claim 1, wherein
the holding element extends in the longitudinal axial direction of the functional element in the compressed state of the functional element.

12. The device as claimed in
claim 1, wherein
a plurality of holding elements are provided and have different lengths.

13. The device as claimed in
claim 1, wherein
at least one annular segment with a holding element or with a plurality of holding elements and at least one annular segment without holding elements are arranged in the longitudinal direction of the lattice structure.

14. The device as claimed in claim 13,
wherein
the ratio of the number of cells of an annular element with holding element to the number of cells of an annular element without holding element is at most 3:4, in particular at most 2:3, at most 1:2, or at most 1:3.

15. The device as claimed in
claim 13, wherein
the ratio of the number of annular elements with at least one holding element to the number of annular elements without holding element is at least 1:3, in particular at least 1:2, at least 2:3, at least 1:1, at least 3:2, at least 2:1, at least 3:1, or at least 4:1.

16. A medical device for removing concretions from hollow organs of the body, with a functional element, which has a rotationally symmetrical lattice structure and a means for holding a concretion in the lattice structure, and a catheter for introducing the functional element into the body and for removing it therefrom, the functional element being convertible from a compressed state in the catheter into an expanded state outside the catheter, in which the functional element is arranged distally from the catheter,
wherein
the lattice structure has a cutting region with webs, which are adapted in such a way that they at least partially radially penetrate the concretion during the conversion of the functional element from the compressed state into the expanded state, and the holding means comprises at least one holding element, which is connected to the lattice structure and also to the guide wire arranged in the catheter in such a way that the holding element is deflected radially inwardly in the expanded state of the functional element.
